(19) United States
(12) Patent Application Publication
(10) Pub. No.: US 2024/0324888 A1
(43) Pub. Date: Oct. 3, 2024

(54) SPORT ASSISTANCE SYSTEM AND SPORT ASSISTANCE METHOD

(71) Applicant: INDUSTRIAL TECHNOLOGY RESEARCH INSTITUTE, Hsinchu (TW)

(72) Inventors: Ming-Chieh TSAI, Hsinchu County (TW); Ching-Yu HUANG, Taoyuan City (TW); Ying-Han HUANG, New Taipei City (TW); Yoong-Kee SEK, Hsinchu City (TW); Po-Ta CHUANG, Taipei City (TW); Yan-Hao HUANG, Hsinchu County (TW)

(73) Assignee: INDUSTRIAL TECHNOLOGY RESEARCH INSTITUTE, Hsinchu (TW)

(21) Appl. No.: 18/621,670

(22) Filed: Mar. 29, 2024

Related U.S. Application Data
(60) Provisional application No. 63/455,269, filed on Mar. 29, 2023.

(51) Int. Cl. A61B 5/024

(52) U.S. Cl. CPC A61B 5/02416

(57) ABSTRACT

A sport assistance system and a sport assistance method are provided. The sport assistance method includes: obtaining a real-time heart rate; executing a first assistance procedure according to a target heart rate, a maximum heart rate, a resting heart rate, a first media and the real-time heart rate; and executing a second assistance procedure according to the target heart rate and the real-time heart rate. The first assistance procedure includes: playing the first media at a first tempo rate; and adjusting the first tempo rate according to the real-time heart rate until the real-time heart rate is close to the target heart rate and a first scheduled time is met. The first initial value is greater than the resting heart rate. The second assistance procedure includes: playing a second media; and adjusting a second tempo rate according to an adjustment parameter until a second scheduled time is met.

### CROSS REFERENCE TO RELATED APPLICATIONS

This non-provisional application claims the benefit under 35 U.S.C. § 119 (e) to U.S. Provisional Application No. 63/455,269, filed on Mar. 29, 2023, all of which are hereby expressly incorporated by reference into the present application.

### BACKGROUND

### Technical Field

The present disclosure relates to a sport assistance system and a sport assistance method, and in particular to a sport assistance system and a sport assistance method that assists in adjusting a real-time heart rate of a user based on media tempo rates to improve sport efficiency.

### Related Art

Keeping the habit of doing sports helps to improve the body's motor ability and stay healthy, and depending on the training objective, the training program can be arranged in a targeted manner. At present,many studies have pointed out that keeping the heart rate within a specific heart rate range during anaerobic training and aerobic training can improve the sport effect. At present, scientific training only considers the influence of the heart rate on the sport effect, but ignores the influence of the user's nervous system on the sport efficiency. For example, when the user performs moderate and high intensity trainings, although the heart rate during exercise is kept in the specified heart rate range, the user's nervous system has not yet reached the corresponding level. This increases the risk of injury to the user and the quality of movement may be affected.

### SUMMARY

In some embodiments, a sport assistance method includes: obtaining a real-time heart rate; executing a first assistance procedure according to a target heart rate, a maximum heart rate, a resting heart rate, a first media and the real-time heart rate; and executing a second assistance procedure according to the target heart rate and the real-time heart rate. The first assistance procedure includes: playing the first media at a first initial value as a first tempo rate of the first media; and adjusting the first tempo rate according to the real-time heart rate until the real-time heart rate is close to the target heart rate and a first scheduled time is met. The first initial value is greater than the resting heart rate. The second assistance procedure includes: playing a second media; and obtaining an adjustment parameter according to a coupling factor, and adjusting a second tempo rate according to the adjustment parameter until a second scheduled time is met.

In some embodiments, the step of adjusting the first tempo rate according to the real-time heart rate until the real-time heart rate is close to the target heart rate and the first scheduled time is met includes: obtaining a correlation coefficient according to the real-time heart rate and the first tempo rate; and executing a tempo rate adjustment procedure in response to that the real-time heart rate is not close to the target heart rate or the first scheduled time is not met, where the tempo rate adjustment procedure includes: continuing the tempo rate adjustment procedure in response to that the correlation coefficient is not smaller than a correlation lower limit and the first scheduled time is not met; increasing the first tempo rate by a scheduled step in response to that the correlation coefficient is smaller than the correlation lower limit and the first scheduled time is not met, and continuing the tempo rate adjustment procedure; continuing the tempo rate adjustment procedure in response to that the first scheduled time is not met and the real-time heart rate is not close to the target heart rate; and terminating the tempo rate adjustment procedure and the first assistance procedure in response to that the real-time heart rate is close to the target heart rate and the first scheduled time is met.

In some embodiments, the step of obtaining the adjustment parameter according to the coupling factor, and adjusting the second tempo rate according to the adjustment parameter until the second scheduled time is met includes: executing a coupling factor determination procedure, the coupling factor determination procedure including: obtaining the coupling factor according to a sport tempo rate and the second tempo rate; continuing the coupling factor determination procedure when the coupling factor is smaller than a coupling factor lower limit; and terminating the coupling factor determination procedure when the coupling factor is not smaller than the coupling factor lower limit; and determining the adjustment parameter according to the second tempo rate, the real-time heart rate, the sport tempo rate and a tempo rate adjustment model.

In some embodiments, the sport assistance method further includes: executing a third assistance procedure according to the maximum heart rate, the resting heart rate, a third media and the real-time heart rate. The third media has a third tempo rate. The third assistance procedure includes: playing the third media at a third initial value as the third tempo rate; and adjusting the third tempo rate according to the real-time heart rate until the real-time heart rate is close to the resting heart rate and a third scheduled time is met.

In some embodiments, the step of adjusting the third tempo rate according to the real-time heart rate until the real-time heart rate is close to the resting heart rate and the third scheduled time is met further includes: obtaining a correlation coefficient according to the real-time heart rate and the third tempo rate; and executing a tempo rate adjustment procedure in response to that the real-time heart rate is not close to the resting heart rate or the third scheduled time is not met, where the tempo rate adjustment procedure includes: continuing the tempo rate adjustment procedure in response to that the correlation coefficient is not smaller than a correlation lower limit and the third scheduled time is not met; decreasing the third tempo rate by a scheduled step in response to that the correlation coefficient is smaller than the correlation lower limit and the third scheduled time is not met, and continuing the tempo rate adjustment procedure; continuing the tempo rate adjustment procedure in response to that the third scheduled time is not met and the real-time heart rate is not close to the resting heart rate; and terminating the tempo rate adjustment procedure and the third assistance procedure in response to that the real-time heart rate is close to the resting heart rate and the third scheduled time is met.

In some embodiments, a sport assistance system includes: a heart rate sensor, configured to obtain a real-time heart rate; and a processor configured to: execute a first assistance procedure according to a target heart rate, a maximum heart rate, a resting heart rate, a first media and the real-time heart rate; and execute a second assistance procedure according to the target heart rate and the real-time heart rate. The first assistance procedure includes: playing the first media at a first initial value as a first tempo rate, the first initial value being greater than the resting heart rate; and adjusting the first tempo rate according to the real-time heart rate until the real-time heart rate is close to the target heart rate and a first scheduled time is met. The second assistance procedure includes: playing a second media, the second media having a second tempo rate; and obtaining an adjustment parameter according to a coupling factor, and adjusting a second tempo rate according to the adjustment parameter until a second scheduled time is met.

Based on the above, in some embodiments, the sport assistance system is configured to selectively execute the first assistance procedure, the second assistance procedure or the third assistance procedure in the sport assistance method. The first assistance procedure is executed when the user just starts to do sports, and at this moment, the real-time heart rate is close to the resting heart rate. The first assistance procedure may guide the user to warm up through the first media, so that the heart rate (real-time heart rate) is increased to the target heart rate in the first scheduled time. The first tempo rate of the first media may be adjusted according to the real-time heart rate, so that the real-time heart rate is gradually increased to the target heart rate under the synchronization of the first tempo rate. The second assistance procedure may guide the user to train through the second media, so that the heart rate (real-time heart rate) is kept at the target heart rate in the second scheduled time and reaches a state of resonance and harmony, thereby improving the sport efficiency and quality of the user. The third assistance procedure may guide the user to recover through the third media, so that the heart rate is decreased to the recovery heart rate in the third scheduled time, and the third tempo rate of the third media can be adjusted according to the real-time heart rate, so that the real-time heart rate is gradually decreased to be close to the resting heart rate under the synchronization of the third tempo rate, thereby reducing the risk of injury.

Various embodiments are provided below for detailed description. However, the embodiments are merely used as examples and will not limit the scope of protection of the present disclosure. In addition, some elements are omitted from the drawings in the embodiments to clearly show the technical characteristics of the present disclosure. The same reference numerals in all drawings will be used to denote the same or similar elements.

### BRIEF DESCRIPTION OF THE DRAWINGS

FIG. 1 is a schematic view of a sport assistance system in some embodiments of the present disclosure;

FIG. 2 is a block diagram of the sport assistance system in some embodiments of the present disclosure;

FIG. 3 is a flowchart of a first assistance procedure of the sport assistance system in some embodiments of the present disclosure;

FIG. 4 is a flowchart of a second assistance procedure of the sport assistance system in some embodiments of the present disclosure;

FIG. 5 is a flowchart of substeps of the first assistance procedure in FIG. 3;

FIG. 6 is a flowchart of substeps of the second assistance procedure in FIG. 4;

FIG. 7 is a flowchart of a third assistance procedure of the sport assistance system in some embodiments of the present disclosure;

FIG. 8 is a flowchart of substeps of the third assistance procedure in FIG. 7;

FIG. 9 is a flowchart of steps of the sport assistance method in some embodiments of the present disclosure;

FIG. 10 is a real-time heart rate graph measured by the sport assistance system in some embodiments of the present disclosure, which shows heart rate positions corresponding to a first tempo rate and a third tempo rate;

FIG. 11 shows waveform diagrams of the sport assistance system before and after adjusting the second tempo rate in some embodiments of the present disclosure;

FIG. 12 is a flowchart of a tempo rate adjustment procedure when the sport assistance system executes the first assistance procedure in some embodiments of the present disclosure;

FIG. 13 is a schematic diagram of output results of the tempo rate adjustment procedure in FIG. 12;

FIG. 14 is a flowchart of a tempo rate adjustment procedure when the sport assistance system executes the third assistance procedure in some embodiments of the present disclosure;

FIG. 15 is a schematic diagram of output results of the tempo rate adjustment procedure in FIG. 14;

FIG. 16 is a flowchart of a first assistance procedure of the sport assistance system in other embodiments of the present disclosure;

FIG. 17 is a flowchart of substeps of the first assistance procedure in FIG. 16;

FIG. 18 is a flowchart of a tempo rate adjustment procedure when the sport assistance system executes the first assistance procedure in other embodiments of the present disclosure;

FIG. 19 is a flowchart of a tempo rate adjustment procedure when the sport assistance system executes the third assistance procedure in other embodiments of the present disclosure;

FIG. 20 is a schematic diagram of output results of the tempo rate adjustment procedure in FIG. 19; and

FIG. 21 is a flowchart of a tempo rate adjustment procedure when the sport assistance system executes the third assistance procedure in other embodiments of the present disclosure.

### DETAILED DESCRIPTION

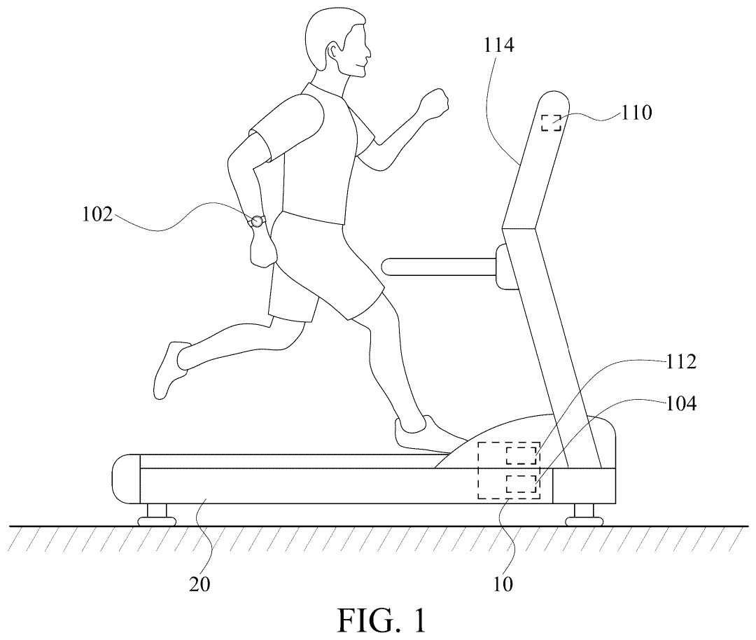

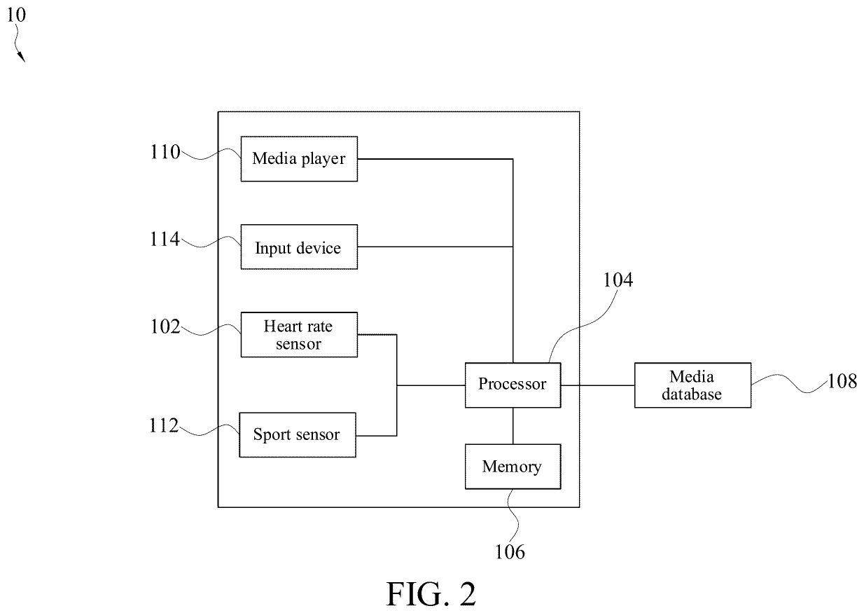

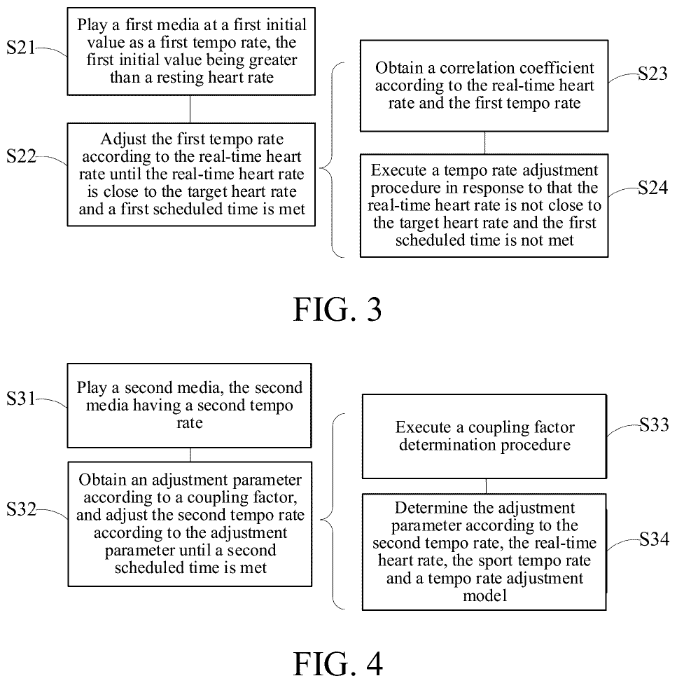

Referring to FIG. 1, FIG. 2, FIG. 3 and FIG. 4, FIG. 1 is a schematic view of a sport assistance system in some embodiments of the present disclosure. FIG. 2 is a block diagram of the sport assistance system in some embodiments of the present disclosure. FIG. 3 is a flowchart of a first assistance procedure of the sport assistance system in some embodiments of the present disclosure. FIG. 4 is a flowchart of a second assistance procedure of the sport assistance system in some embodiments of the present disclosure. As shown in FIG. 1 to FIG. 4, the sport assistance system 10 includes a heart rate sensor 102 and a processor 104. The heart rate sensor 102 is configured to obtain a real-time heart rate. The processor 104 is configured to: execute a first assistance procedure according to a target heart rate, a maximum heart rate, a resting heart rate, a first media and the real-time heart rate; and execute a second assistance procedure according to the target heart rate and the real-time heart rate (detailed later).

As shown in FIG. 1, the sport assistance system 10 may be applied to a sport apparatus 20. The sport apparatus 20 may be, for example, a treadmill, a flywheel bike, or a cross trainer. The sport apparatus 20 shown in FIG. 1 is a treadmill. The following description will be made in an example of a treadmill. When a user trains with the sport apparatus 20, the sport assistance system 10 may provide a corresponding media to guide the user's real-time heart rate and movements in different phases during exercise. For example, when the user is in a warm-up phase, the sport assistance system 10 executes the first assistance procedure to play the first media so as to assist the user in efficiently increasing the heart rate to the target heart rate; when the user is in a training phase, the sport assistance system 10 executes the second assistance procedure to play the second media so as to assist the user in keeping the heart rate at the target heart rate; and when the user is in a recovery phase, the sport assistance system 10 executes the third assistance procedure to play a third media so as to assist the user in efficiently decreasing the heart rate close to the resting heart rate. During implementation, the user may selectively make the sport assistance system 10 execute one or more of the first assistance procedure, the second assistance procedure and the third assistance procedure.

The heart rate sensor 102 may be configured to measure the real-time heart rate of the user. The heart rate sensor 102 may be fixed to the sport apparatus 20 or worn on the user to measure the heart rate of the user. In an example of being fixed to the sport apparatus 20, the heart rate sensor 102 may be an electrode sensor, configured to detect a change rate of a myocardial potential of the user to obtain the heart rate. In an example of being worn on the user, the heart rate sensor 102 may be a photoplethysmography (PPG) sensor, configured to obtain a photoplethysmography signal and obtain the heart rate according to the photoplethysmography signal. The heart rate sensor 102 may also be a clip-type heart rate sensor, which senses the change of flow of blood in the capillaries of an earlobe or finger with each heartbeat by using far infrared rays so as to obtain the heart rate.

The processor 104 is configured to process and execute the procedures (e.g., the first assistance procedure, the second assistance procedure and the third assistance procedure, which will be detailed later) of the embodiment and signals (e.g., the real-time heart rate, the first media, the second media, the third media and a sport tempo rate). The processor 104 may be implemented by one or more processing elements. Each processing element may be, for example, a microprocessor, a microcontroller, a digital signal processor (DSP), a central processing unit (CPU), a programmable logic controller (PLC), an application specific integrated circuit (ASIC), a field programmable gate array (FPGA), or a finite-state machine (FSM). The processor 104 may also be implemented by an integrated circuit (IC), or a system-on-a-chip (SoC).

The target heart rate may be a heart rate value set by the user according to a sport objective. The target heart rate may also be set according to a training objective, for example, a heart rate range of aerobic exercise or anaerobic exercise. The target heart rate may be determined based on a heart rate reserve method. The target heart rate may be the resting heart rate+the heart rate reserve*a first multiplier. The heart rate reserve is the maximum heart rate−the resting heart rate. For example, in consideration of the intensity of the sport (moderate intensity sport), the first multiplier of the target heart rate may be set to 50% (50% may correspond to moderate and high intensity sports). For example, in a case that the maximum heart rate is 180 bpm, the resting heart rate is 80 bpm and the first multiplier is set to 50%, the target heart rate is 80 bpm+ (180 bpm-80 bpm)*50%=130 bpm.

The maximum heart rate may be set according to personal sport data or history heart rate data of the user. For example, the personal sport data includes age, and the maximum heart rate may be obtained by 220—age. However, the calculation manner is not limited thereto.

The resting heart rate is the heart rate of the user before doing sports, which may be obtained from the user data or may be the real-time heart rate measured by the heart rate sensor 102 before the user does sports. The resting heart rate may also be a preset heart rate value (e.g., any value from 60 bpm to 100 bpm may be taken as the resting heart rate).

The first media has a first tempo rate, the second media has a second tempo rate, and the third media has a third tempo rate. The first media, the second media and the third media may each be a sound signal (e.g., a beat sound or a remix), a visible light signal (e.g., a rhythmic flash), a music or an audio-visual film. In some embodiments, as shown in FIG. 1, the processor 104 may play the first media, the second media and the third media (detailed later). In the embodiment where the first media, the second media and the third media are sound signals, the first tempo rate, the second tempo rate and the third tempo rate are the beat sounds of the sound signals. In the embodiment where the first media, the second media and the third media are the visible light signals, the first tempo rate, the second tempo rate and the third tempo rate are flicker frequencies of visible light or switching frequencies between strong light and weak light. It should be noted that the real-time heart rate may correspond to the tempo rate of the media. Specifically, the first tempo rate, the second tempo rate, the third tempo rate and the real-time heart rate are all measured in beats per minute (bpm), and the first tempo rate, the second tempo rate and the third tempo rate may correspond to the human heart rate variation range. The processor 104 may play the first media, the second media and the third media respectively at the first tempo rate, the second tempo rate and the third tempo rate so as to guide the user to do sports respectively according to the first tempo rate, the second tempo rate and the third tempo rate. Furthermore, the processor 104 may adjust the first tempo rate when playing the first media at the first tempo rate (executing the first assistance procedure) so as to guide the real-time heart rate of the user to increase. Similarly, the processor 104 may also adjust the second tempo rate and the third tempo rate when executing the second assistance procedure and the third assistance procedure, which will be detailed later. It should be noted that the processor 104 adjusting the first, second and third tempo rates may mean that the processor 104 adjusts the playback rate of the media to lengthen or shorten the interval between consecutive beats. In the example of the first assistance procedure, the processor 104 will gradually increase the first tempo rate, i.e., increase the playback rate of the first media. In the example of the second assistance procedure, the processor 104 will gradually increase the second tempo rate, i.e., increase the playback rate of the second media. In the example of the third assistance procedure, the processor 104 will gradually decrease the third tempo rate, i.e., decrease the playback rate of the third media.

In some embodiments, the first media, the second media and the third media may have the same media signal or different media signals. The above description is the case where the first media, the second media and the third media have different media signals. The first media may have the media signal that is more in line with a warm-up atmosphere, the second media may have the media signal with a training or exercise atmosphere, and the third media may have the media signal with a recovery atmosphere. In the embodiment where the first media, the second media and the third media have the same media signal, the processor 104 may play the media signal according to the required tempo rate when executing the first, second and third assistance procedures.

In some embodiments, as shown in FIG. 2, the first media, the second media and the third media may be stored in a memory 106. The processor 104 may obtain the first media, the second media and/or the third media from the memory 106. The memory 106 may be, but not limited to, a volatile memory (such as a random access memory (RAM)), a volatile memory (such as a read only memory (ROM)), a hard drive or a solid-state drive. The memory 106 may be used for storing a plurality of real-time heart rates continuously measured by the heart rate sensor 102. In some embodiments, the first media and the second media may also be a streaming media. The sport assistance system 10 may be connected to a media database 108 through a network. The first media and the second media are stored in the media database 108, and the sport assistance system 10 may download the first media and the second media from the media database 108.

As shown in FIG. 1 and FIG. 2 again, in some embodiments, the sport assistance system 10 further includes a media player 110. The media player 110 may be a speaker. The media player 110 shown in FIG. 1 is fixed to the sport apparatus 20. When the user operates the sport apparatus 20, the media player 110 may play the first media, the second media or the third media. The media player 110 may also be a wearable device (e.g., a wireless headset or a wired headset), a display device or a lighting device. When the media player 110 is implemented as a display device, the display device may change its display (e.g., color block or animation) according to the tempo rate of the media.

As shown in FIG. 1 and FIG. 2 again, in some embodiments, the sport assistance system 10 further includes a sport sensor 112. The sport sensor 112 may be, for example, a triaxial accelerometer, a triaxial gravity sensor, a cadence sensor, a rotary encoder or an optical sensor. The sport tempo rate may refer to the frequency at which the user completes motions per unit time measured by the sport sensor 112. In an example where the sport apparatus 20 is a treadmill and the sport sensor 112 is a cadence sensor in FIG. 1, the sport sensor 112 is fixed to the sport apparatus 20, and may detect the cadence of the user as the sport tempo rate of the second assistance procedure. In an example where the sport apparatus 20 is a flywheel bike and the sport sensor 112 is a rotary encoder, the rotary encoder may detect the frequency (revolutions) per unit time of the user stepping on the pedal as the sport tempo rate of the second assistance procedure.

As shown in FIG. 1 and FIG. 2 again, in some embodiments, the sport assistance system 10 further includes an input device 114. The input device 114 may be, for example, a keyboard, a computer, a touch panel or a mobile communication device. The user may input the target heart rate or the personal sport data through the input device 114.

As shown in FIG. 1, FIG. 2, FIG. 3 and FIG. 4 again, in some embodiments, the processor 104 may selectively execute the first assistance procedure or the second assistance procedure according to the real-time heart rate. The processor 104 may execute the first assistance procedure at the beginning, and then execute the second assistance procedure when the real-time heart rate is close to the target heart rate and a duration of the first assistance procedure is not smaller than a first scheduled time (the first scheduled time may refer to a warm-up time). In some embodiments, the processor 104 may execute the first assistance procedure when receiving an initial signal. The initial signal may be sent by operating the input device 114 by the user. The processor 104 may also execute the first assistance procedure according to a time point of the user in the warm-up phase. Besides, when the processor 104 initially executes the first assistance procedure, a value slightly higher than the resting heart rate is used as the first tempo rate of the first media, and when a first scheduled time (warm-up time) is met and the real-time heart rate is close to the target heart rate, the second assistance procedure is executed.

The steps of the first, second and third assistance procedures executed by the processor 104 will be described below.

As shown in FIG. 3, the first assistance procedure includes the following steps:

Step S21: playing the first media at a first initial value as a first tempo rate, the first initial value being greater than the resting heart rate; and

Step S22: adjusting the first tempo rate according to the real-time heart rate until the real-time heart rate is close to the target heart rate and a first scheduled time is met.

The “playing the first media at a first initial value as a first tempo rate” in step S21 may mean that at the beginning of the warm-up phase of the sporter, the processor 104 sets the first initial value of the first tempo rate to a value slightly greater than the resting heart rate (between the resting heart rate and the target heart rate, detailed later) at the beginning of the first assistance procedure in order to guide the heart rate of the user to efficiently increase to the target heart rate through the first media. The first initial value refers to the initial value (tempo rate) of the first tempo rate. By setting the first initial value to be slightly greater than the resting heart rate, when the user feels (hears sound with ears or senses light with eyes) the first tempo rate of the first media and does sports along with the first media, the movements of the user are accelerated under the guide of the first tempo rate (the direct connection between auditory neurons and motor neurons makes the first media excite the parasympathetic nerves of the user). In some embodiments, the first initial value may be the resting heart rate+the heart rate reserve*a second multiplier. The first multiplier is greater than the second multiplier. The second multiplier may be 20%, and then the first initial value is the resting heart rate+20%*(the maximum heart rate—the resting heart rate). For example, in a case that the maximum heart rate is 180 bpm and the resting heart rate is 80 bpm, the first initial value is 100 bpm (80+ (180−80)*20%).

The “adjusting the first tempo rate according to the real-time heart rate until the real-time heart rate is close to the target heart rate and a first scheduled time is met” in step S22 may mean that as the user does sports under the guide of the first media, when the real-time heart rate exceeds the first tempo rate, the first media may guide the heart rate of the user less effectively or ineffectively. Therefore, during the execution of the first assistance procedure, the processor 104 may increase the first tempo rate (the first tempo rate is equal to or greater than the real-time heart rate) in multiple phases during the increase of the first tempo rate to keep the increasing effect of the real-time heart rate (detailed later). It should be noted that after the processor 104 adjusts the first tempo rate, if the real-time heart rate is increased to the first tempo rate, the user can maintain an efficient heart rate increase rate. This helps the user quickly get into the training phase from the warm-up phase in an appropriate training environment. “The real-time heart rate is close to the target heart rate” means that the real-time heart rate is 90% to 110% of the target heart rate. The first scheduled time may be a preset minimum warm-up time, for example, 5 min, 6 min, and 10 min, and the first scheduled time may be determined according to the motor ability of the user.

In some embodiments, when the first assistance procedure is terminated, the first tempo rate is a first termination value, and the first termination value is close to the tempo rate corresponding to the target heart rate. Specifically, when the first tempo rate is close to or exceeds the target heart rate, it indicates that the user may continue the training phase from the warm-up phase. In other words, the processor 104 will end the first assistance procedure and start the second assistance procedure. When the processor 104 adjusts the first tempo rate, the interval between the first initial value and the first termination value may be divided into at least one stage, and the processor 104 may increase the first tempo rate at each stage (detailed later). In some embodiments, the first termination value is substantially equal to the target heart rate (e.g., the target heart rate is set at 130 bpm, and the first termination value is 130 bpm).

As shown in FIG. 4, the second assistance procedure includes the following steps:

Step S31: playing a second media, the second media having a second tempo rate;
and

Step S32: obtaining an adjustment parameter according to a coupling factor, and adjusting the second tempo rate according to the adjustment parameter until a second scheduled time is met.

In the “playing a second media, the second media having a second tempo rate” in step S31, the second media may be the first media, and the second tempo rate may be the first termination value of the first tempo rate (i.e., the first tempo rate when the first assistance procedure is terminated). The second tempo rate may be set according to the target heart rate. When executing the second assistance procedure, the processor 104 may search for the second media with the corresponding tempo rate according to the tempo rate of the target heart rate and play the second media, or play the first media at a second initial value as the second tempo rate (the first media and the second media have the same media signal). However, the second initial value of the second tempo rate is greater than the first initial value of the first tempo rate. The second initial value being greater than the first initial value may mean that the processor 104 play the second media at a higher playback rate (e.g., at a playback rate higher than 1×).

In the “obtaining an adjustment parameter according to a coupling factor, and adjusting the second tempo rate according to the adjustment parameter until a second scheduled time is met” in step S32, the processor 104 may preset the second scheduled time (the second scheduled time may refer to the training time). The second scheduled time may be set according to the type of the sport, the training objective or the training program. For example, the training program of the user records riding a bike for one hour and keeping the heart rate at 130 bpm. The user may input the target heart rate and the second scheduled time through the sport assistance system 10, and the processor 104 may track whether the real-time heart rate is kept at the target heart rate and the second scheduled time is met. It should be noted that when the heart rate is kept at the target heart rate, the user can achieve a better training effect. When the second tempo rate is coupled with the body movements and real-time heart rate of the user, the cardiolocomotor synchronization can operate efficiently. In this way, the performance of the user in repetitive body movements can be improved, the attention of the user during training can be improved, and the risk of injury can be reduced. For example, if the real-time heart rate is kept at the target heart rate and the intensity of the sport (e.g., the cadence of the bike) is not decreased, but the second tempo rate is not coupled with the movements and the real-time heart rate, the performance of the second media guiding the training of the user may be not as effective as expected. Therefore, when executing the second assistance procedure, the processor 104 may adjust the second tempo rate according to the current movement state of the user by using the adjustment parameter (the process of the processor 104 determining the adjustment parameter will be detailed later), so that the movements and real-time heart rate of the user are coupled with the second tempo rate, thereby improving the training performance. The coupling factor may refer to the synchronization between the movements of the user and the second tempo rate when the user is in the training phase.

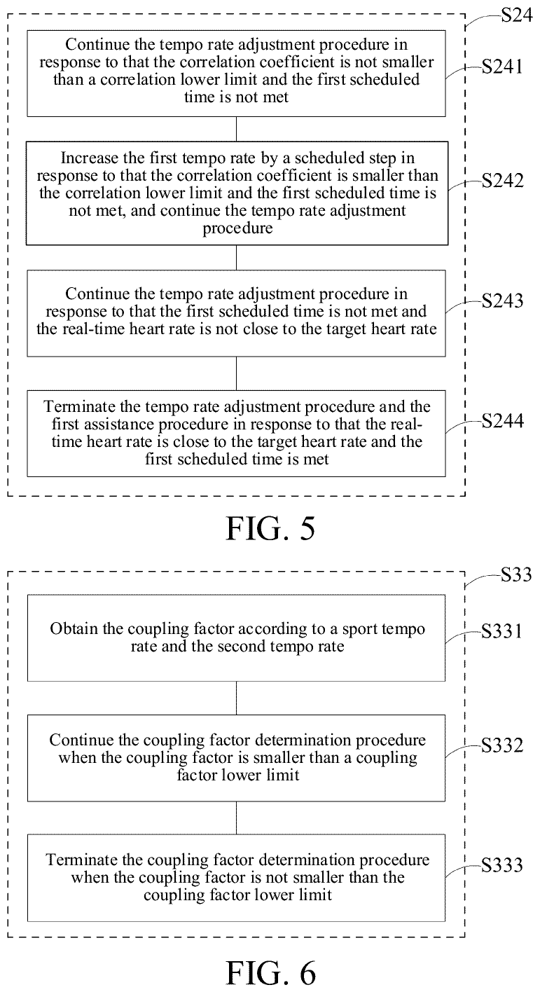

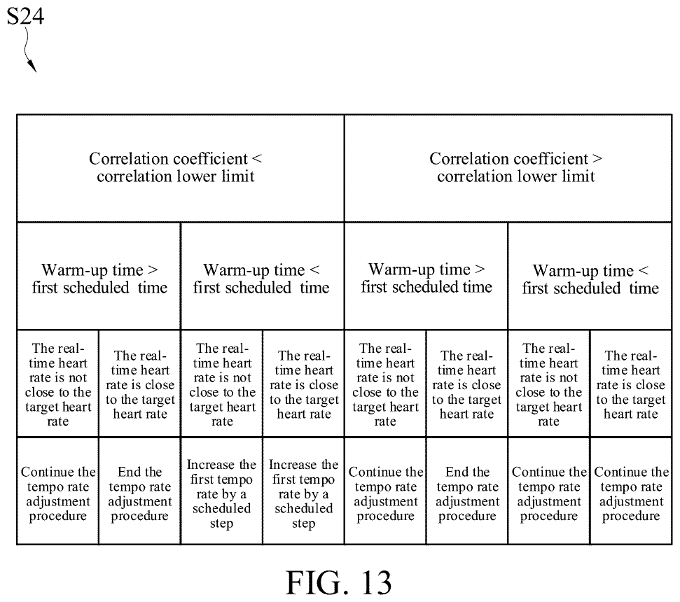

Referring to FIG. 2, FIG. 3, FIG. 5, FIG. 12 and FIG. 13, FIG. 5 is a flowchart of substeps of the first assistance procedure in FIG. 3. FIG. 12 is a flowchart of a tempo rate adjustment procedure when the sport assistance system executes the first assistance procedure in some embodiments of the present disclosure. FIG. 13 is a schematic diagram of output results of the tempo rate adjustment procedure in FIG. 12. In some embodiments, the “adjusting the first tempo rate according to the real-time heart rate until the real-time heart rate is close to the target heart rate and a first scheduled time is met” in step S22 in the first assistance procedure includes:

Step S23: obtaining a correlation coefficient according to the real-time heart rate and the first tempo rate; and

Step S24: executing a tempo rate adjustment procedure in response to that the real-time heart rate is not close to the target heart rate or the first scheduled time is not met, where the tempo rate adjustment procedure includes:

Step S241: continuing the tempo rate adjustment procedure in response to that the correlation coefficient is not smaller than a correlation lower limit and the first scheduled time is not met;

Step S242: increasing the first tempo rate by a scheduled step in response to that the correlation coefficient is smaller than the correlation lower limit and the first scheduled time is not met, and continuing the tempo rate adjustment procedure;

Step S243: continuing the tempo rate adjustment procedure in response to that the first scheduled time is not met and the real-time heart rate is not close to the target heart rate; and

Step S244: terminating the tempo rate adjustment procedure and the first assistance procedure in response to that the real-time heart rate is close to the target heart rate and the first scheduled time is met.

The “obtaining a correlation coefficient according to the real-time heart rate and the first tempo rate” in step S23 may mean that the processor 104 obtains the correlation coefficient between the real-time heart rate and the first tempo rate in a sliding time window. The correlation coefficient may indicate whether the real-time heart rate is synchronized with the first tempo rate. In implementation, the correlation coefficient may be Pearson product-moment correlation coefficient, Kendall's tau coefficient and Spearman's correlation coefficient.

The following is a description in an example where the correlation coefficient is the Pearson product-moment correlation coefficient. The aforementioned time window may be 60 s. In an example where the processor 104 obtains the first tempo rate and the real-time heart rate at a sampling frequency of 1 time/s, the processor 104 can obtain 60 first tempo rates and 60 real-time heart rates in the time window 60 s, which are put into the following Pearson product-moment correlation coefficient formula (formula (1) below) to obtain the correlation coefficient.

$$r ( x , y ) = x = \frac{{\sum }_{i = 1}^{n} ( H {R}_{i} - \overline{HR} ) ( M {T}_{i} - \overline{MT} )}{\sqrt{{\sum }_{i = 1}^{n} {( H {R}_{i} - \overline{HR} )}^{2} \sqrt{{\sum }_{i = 1}^{n} {( M {T}_{i} - \overline{MT} )}^{2}}}} \qquad formula ( 1 )$$

where r is the correlation coefficient;
n is the time coefficient of the time window;
HR is the real-time heart rate; and
MT is the tempo rate of the media.

It should be noted that the parameter n is the time coefficient of the time window. If n is set to 60 s, the processor 104 retrieves the real-time heart rates and the tempo rates (the first tempo rates in the first assistance procedure, and the third tempo rates in the third assistance procedure) of the media from the 1^{st} second to the 59^{th} second.

When the correlation coefficient (Pearson product-moment correlation coefficient) is 1, it indicates that the first tempo rate is positively correlated with the real-time heart rate. When the correlation coefficient is smaller than 1 but close to 1 (e.g., 0.8), it indicates that the first tempo rate is highly correlated with the real-time heart rate.

The processor 104 may obtain the correlation coefficient in the sliding time window. This may mean that the processor 104 obtains the correlation coefficient corresponding to 10:04:00 to 10:04:59 at 10:05:00, and then obtains the correlation coefficient corresponding to 10:04:01 to 10:05:00 at 10:05:01 (which is an example of obtaining a correlation coefficient every second), and so on. In some embodiments, the processor 104 obtains a correlation coefficient every minute, every 30 seconds, and every 20 seconds.

In step S24, after the processor 104 obtains the correlation coefficient, if the real-time heart rate at this moment has not reached the target heart rate and the sport time has not reached the first scheduled time, the processor 104 executes the tempo rate adjustment procedure.

In step S241, the “continuing the tempo rate adjustment procedure in response to that the correlation coefficient is not smaller than a correlation lower limit and the first scheduled time is not met” may mean that after obtaining the correlation coefficient, the processor 104 compares the correlation coefficient with the correlation lower limit. If the correlation coefficient is equal to or greater than the correlation lower limit and the sport time (from the start of the first assistance procedure to the current time at the comparison of the correlation coefficient) does not meet the first scheduled time, the processor 104 continues the tempo rate adjustment procedure (i.e., repeats the determination process of step S241). The correlation lower limit may be 0.8. In step S241, when the correlation coefficient is greater than 0.8, it indicates that the real-time heart rate is nearly synchronized with the first tempo rate. However, when the sport time does not meet the first scheduled time, it indicates that the warm-up time of the user is not enough. Therefore, in this case, the processor 104 will not execute the second assistance procedure, but will continue the tempo rate adjustment procedure.

The “increasing the first tempo rate by a scheduled step in response to that the correlation coefficient is smaller than the correlation lower limit and the first scheduled time is not met, and continuing the tempo rate adjustment procedure” in step S242 may mean that in a case that the processor 104 determines that the correlation coefficient is smaller than the correlation lower limit and the sport time does not meet the first scheduled time, the processor 104 will increase the first tempo rate by the scheduled step at this moment. It should be noted that when the processor 104 executes step S242 for the first time, the first tempo rate is the first initial value. When the processor 104 executes step S242 next time, the previous first tempo rate is increased by the scheduled step. Therefore, every time the processor 104 executes step S242, the first tempo rate may be increased by the scheduled step, to increase the playback rate of the first media, so as to guide the user to increase the heart rate when doing sports. The scheduled step may be any tempo rate in a tempo rate range (5 bpm to 20 bpm). The scheduled step may also be obtained based on a third multiplier of the heart rate reserve or the resting heart rate. The third multiplier is an arithmetic progression between the first multiplier and the second multiplier, so that a plurality of steps are formed between the first initial value and the first termination value. For example, in an example where the first initial value is 20% of the heart rate reserve and the first termination value is 50% of the heart rate reserve, the first tempo rate is increased each time by a scheduled step of 25%*the heart rate reserve, 30%*the heart rate reserve, 35%*the heart rate reserve, 40%*the heart rate reserve, 45%*the heart rate reserve and 50%*the heart rate reserve.

Referring to FIG. 10, FIG. 10 is a real-time heart rate graph measured by the sport assistance system in some embodiments of the present disclosure, which shows heart rate positions corresponding to a first tempo rate and a third tempo rate. As shown in FIG. 10, the waveform in the figure is a real-time heart rate HR measured by the heart rate sensor 102. The following is a description in an example where the first initial value is the resting heart rate+20%*the heart rate reserve (100 bpm), the target heart rate is the resting heart rate+50%*the heart rate reserve (130 bpm), the update frequency of the first tempo rate is once per minute, and the scheduled step is set to the heart rate reserve*5%. The target heart rate is marked at a target heart rate position HRₜ in the figure, and the resting heart rate is marked at a resting heart rate position HRₜ in the figure, so that the changes of the real-time heart rate HR and the media tempo rate can be observed. When the first tempo rate is increased for the first time, the first tempo rate is increased from the resting heart rate+20%*the heart rate reserve to the resting heart rate+25%*the heart rate reserve (as shown in FIG. 10, to the tempo rate t11 in the first stage), i.e., from 100 bpm to 105 bpm (80 bpm+ (180 bpm-80 bpm)*25%=105 bpm). When the first tempo rate is increased for the second time, the first tempo rate is increased from the resting heart rate+25%*the heart rate reserve to the resting heart rate+30%*the heart rate reserve (as shown in FIG. 10, to the tempo rate t12 in the second stage), i.e., the first tempo rate is increased from 105 bpm to 110 bpm. When the first tempo rate is increased for the third time, the first tempo rate is increased from the resting heart rate+30%* the heart rate reserve to the resting heart rate+35%*the heart rate reserve (as shown in FIG. 10, to the tempo rate t13 in the third stage), i.e., the first tempo rate is increased from 110 bpm to 115 bpm. When the first tempo rate is increased for the fourth time, the first tempo rate is increased from the resting heart rate+35%*the heart rate reserve to the resting heart rate+40%*the heart rate reserve (as shown in FIG. 10, to the tempo rate t14 in the fourth stage), i.e., the first tempo rate is increased from 115 bpm to 120 bpm. When the first tempo rate is increased for the fifth time, the first tempo rate is increased from the resting heart rate+40%*the heart rate reserve to the resting heart rate+45%*the heart rate reserve (as shown in FIG. 10, to the tempo rate t15 in the fifth stage), i.e., the first tempo rate is increased from 120 bpm to 125 bpm. When the first tempo rate is increased for the sixth time, the first tempo rate is increased from the resting heart rate+45%*the heart rate reserve to the resting heart rate+50%*the heart rate reserve (as shown in FIG. 10, to the tempo rate t16 in the sixth stage), i.e., the first tempo rate is increased from 125 bpm to 130 bpm. In this example, the target heart rate is set to 130 bpm. If the real-time heart rate HR has been close to the target heart rate before the first tempo rate is increased for the fifth time and the first scheduled time has been met, the processor 104 will not increase the first tempo rate for the fifth time, and the processor 104 will terminate the first assistance procedure and start the second assistance procedure. Besides, when the first initial value is increased to the first termination value according to the scheduled step, if the first tempo rate is updated at a frequency of once per minute, it is ensured that the first media can be played for six minutes before the processor 104 executes the second assistance procedure. In other words, the user has a warm-up time of at least six minutes in the warm-up phase. Thereby, the first media has enough time to guide the user to progressively increase the sport speed and heart rate response, so that the user can warm up adequately until the real-time heart rate HR reaches the target heart rate.

The “continuing the tempo rate adjustment procedure in response to that the first scheduled time is not met and the real-time heart rate is not close to the target heart rate” in step S243 may mean that when the sport time does not meet the first scheduled time and the real-time heart rate is not close to the target heart rate, the processor 104 will repeatedly execute step S241 to determine whether the correlation coefficient is smaller than the correlation lower limit and selectively execute step S242 to increase the first tempo rate, thereby ensuring that the first tempo rate is greater than the real-time heart rate.

The “terminating the tempo rate adjustment procedure and the first assistance procedure in response to that the real-time heart rate is close to the target heart rate and the first scheduled time is met” in step S244 may mean that when determining that the real-time heart rate is equal to or greater than the target heart rate and the sport time meets the first scheduled time, the processor 104 terminates the tempo rate adjustment procedure and the first assistance procedure and executes the second assistance procedure.

“The real-time heart rate is close to the target heart rate” may mean that the processor 104 determines whether the difference between the real-time heart rate and the target heart rate (the target heart rate—the real-time heart rate) is smaller than an error threshold. If the difference between the real-time heart rate and the target heart rate is greater than the error threshold, the processor 104 determines that the real-time heart rate is not close to the target heart rate. On the contrary, if the difference between the real-time heart rate and the target heart rate is smaller than the error threshold, the processor 104 determines that the real-time heart rate is close to the target heart rate. In some embodiments, the error threshold is set to 5%.

In some embodiments, the “increasing the first tempo rate by a scheduled step in response to that the correlation coefficient is smaller than the correlation lower limit and the first scheduled time is not met, and continuing the tempo rate adjustment procedure” in step S242 further includes:

Step S245: adjusting the first tempo rate according to an adjustment step in response to that the first tempo rate lags behind the real-time heart rate, and continuing the tempo rate adjustment procedure.

The “in response to that the first tempo rate lags behind the real-time heart rate” in step S245 may mean that the processor 104 obtains an average of the real-time heart rates (hereinafter referred to as the real-time heart rate average) and another average of the first tempo rates (hereinafter referred to as the first tempo rate average) according to all the real-time heart rates and first tempo rates in the time window. When the first tempo rate average is smaller than the real-time heart rate average, it indicates that the first tempo rate lags behind the real-time heart rate. On the contrary, when the first tempo rate average is equal to or greater than the real-time heart rate average, it indicates that the first tempo rate exceeds the real-time heart rate.

The “adjusting the first tempo rate according to an adjustment step, and continuing the tempo rate adjustment procedure” in step S245 may mean that when determining that the first tempo rate lags behind or exceeds the real-time heart rate, the processor 104 selectively adjusts the first tempo rate. It should be noted that if the correlation coefficient is smaller than the correlation lower limit and the first tempo rate lags behind the real-time heart rate, it indicates that the real-time heart rate is increased at a rate more than expected, so that the first tempo rate is not synchronized with the real-time heart rate. The processor 104 may increase the first tempo rate by the adjustment step (i.e., the first tempo rate+the scheduled step+the adjustment step). On the contrary, if the correlation coefficient is smaller than the correlation lower limit and the first tempo rate exceeds the real-time heart rate, it indicates that the real-time heart rate is increased at a rate less than expected, so that the first tempo rate is not synchronized with the real-time heart rate. The processor 104 may decrease the first tempo rate by the adjustment step (i.e., the first tempo rate+the scheduled step−the adjustment step). In this way, the first tempo rate can be gradually synchronized with the real-time heart rate. The adjustment step may be 5% of the real-time heart rate average. For example, if the first tempo rate before adjustment is 110 bpm and the real-time heart rate average is 112 (the first tempo rate lags behind the real-time heart rate), the first tempo rate after adjustment is 115.6 bpm (110 bpm+112 bpm*5%). For another example, if the first tempo rate before adjustment is 110 bpm and the real-time heart rate average is 105 (the first tempo rate is ahead of the real-time heart rate), the first tempo rate after adjustment is 104.75 bpm (110 bpm-105 bpm*5%). It should be noted that if the first tempo rate is still smaller than the real-time heart rate after the fine adjustment, the first tempo rate may exceed the real-time heart rate in the next scheduled step, so as to ensure that the first heart rate exceeds the real-time heart rate within the warm-up time. Alternatively, if the correlation coefficient is smaller than the correlation lower limit, the processor 104 may perform fine adjustment again (execute step S245 again) until the correlation coefficient is smaller than the correlation lower limit.

Referring to FIG. 2, FIG. 4 and FIG. 6, FIG. 6 is a flowchart of substeps of the second assistance procedure in FIG. 4. In some embodiments, the “obtaining the adjustment parameter according to the coupling factor, and adjusting the second tempo rate according to the adjustment parameter until the second scheduled time is met” in step S32 further includes:

Step S33: executing a coupling factor determination procedure, where the coupling factor determination procedure includes:

Step S331: obtaining a coupling factor according to a sport tempo rate and the second tempo rate;

Step S332: continuing the coupling factor determination procedure when the coupling factor is smaller than a coupling factor lower limit; and

Step S333: terminating the coupling factor determination procedure when the coupling factor is not smaller than the coupling factor lower limit; and

Step S34: determining the adjustment parameter according to the second tempo rate, the real-time heart rate, the sport tempo rate and a tempo rate adjustment model.

The “obtaining the coupling factor according to the sport tempo rate and the second tempo rate” in step S331 may mean that the processor 104 transforms the second tempo rate to a phase plane to obtain a first phase angle; transforms the sport tempo rate to the phase plane to obtain a second phase angle; and obtains the coupling factor according to the first phase angle, the second phase angle and a coupling factor formula. In some embodiments, the coupling factor formula is ∥F1+F2∥/∥F1∥+∥F2∥. F1 is the first phase angle. F2 is the second phase angle. ∥F1+F2∥ is the size of the sum of the first phase angle and the second phase angle. ∥F1∥ is the size of the first phase angle. ∥F2∥ is the size of the second phase angle. ∥F1∥+∥F2∥ is equal to 2.

According to the calculation result of the coupling factor formula, the coupling factor falls within the range of 0 to 1. The closer the coupling factor is to 1, the better the coupling factor between the sport tempo rate and the second tempo rate is. On the contrary, the closer the coupling factor is to 0, the worse the coupling factor between the sport tempo rate and the second tempo rate is. When the user is in the training phase, in order to ensure that the real-time heart rate, the sport tempo rate and the second tempo rate reach a state of resonance and harmony, the coupling factor should be greater than the coupling factor lower limit. The coupling factor lower limit may be 80%. If the coupling factor is equal to or greater than 80%, it indicates that the real-time heart rate, the sport tempo rate and the second tempo rate reach the state of resonance and harmony, and the processor 104 terminates the coupling factor determination procedure (step S333). On the contrary, if the coupling factor is smaller than 80%, it indicates that the real-time heart rate, the sport tempo rate and the second tempo rate may not reach the state of resonance and harmony, and the processor 104 continues the coupling factor determination procedure (step S332).

In some embodiments, the tempo rate adjustment model in step S34 may be a Kuramoto model. The Kuramoto model includes formulae (3) and (4) as follows:

$${θ}_{t + 1 , m e n} = {ω}_{t , men} + {k}_{m e n} sin ( {θ}_{t , music} - {θ}_{t , men} ) \qquad formula ( 3 )$$

$${θ}_{t + 1 , m u s i c} = {ω}_{t , music} + {k}_{m u s i c} sin ( {θ}_{t , men} - {θ}_{t , music} ) \qquad formula ( 4 )$$

where θ_{t+1,men} is an angular speed of the sport tempo rate at time t+1;
θ_{t+1,music} is an angular speed of the second tempo rate at time t+1;
θ_{t,men} is a phase angle of the sport tempo rate at time t;
θ_{t,music} is a phase angle of the second tempo rate at time t;
ω_{t,men} is an initial angular speed of the sport tempo rate at time t;
ω_{t,music} is an initial angular speed of the second tempo rate at time t;
kₘₑₙ is a coupling constant of the sport tempo rate; and
k_{music} is a coupling constant of the second tempo rate.

The first tempo rate (music rhythm) of the first media is first set to experience time t, and the speed and phase angle of the user are calculated and put into formula (4) to obtain θ_{1+1,music}. After time t, the foregoing calculation process is repeated until |θ_{t+1,music}−θ_{t+1,men}|<c, thereby obtaining the coupling factor.

The aforementioned parameters kₘₑₙ and ω_{t,men} may be obtained through a preprocessing procedure. The preprocessing procedure includes:

For the first minute, the user walks at the most natural walking speed, and the system calculates the walking speed, which is ω_{t,men}.

For the second minute, the system generates, according to the speed ω_{t,men}, a tempo rate which is 20% higher than ω_{t,men}, so that the user does sports with this tempo rate.

For the third minute, the system calculates the speed of the user, and calculates kₘₑₙ according to the Kuramoto model.

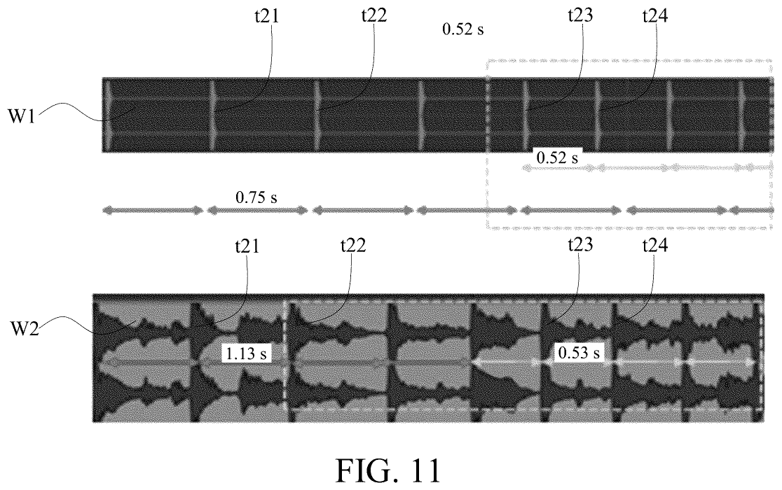

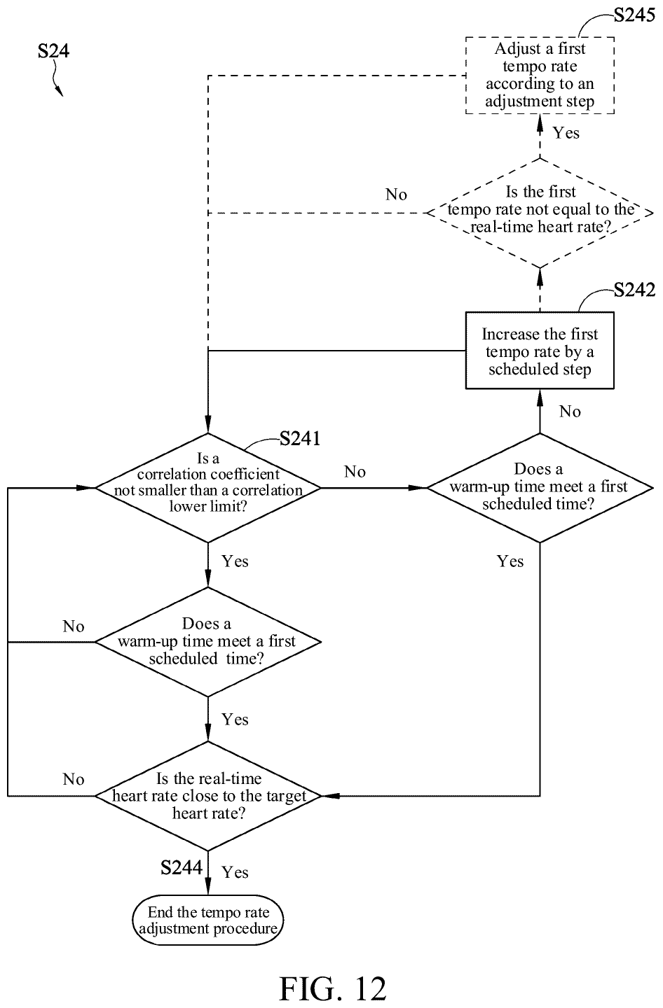

Referring to FIG. 11, FIG. 11 shows waveform diagrams of the sport assistance system before and after adjusting the second tempo rate in some embodiments of the present disclosure. As shown in FIG. 11, in some embodiments, the processor 104 may execute a media editing tool (e.g., an audio time stretching tool) to adjust the second media. The processor 104 may input the original second media and the adjustment parameter calculated by the tempo rate adjustment model into the media editing tool, so that the media editing tool can adjust the second tempo rate of the second media according to the adjustment parameter. As shown in FIG. 11, the interval between a first beat t21 and a second beat t22 of the unadjusted media waveform W1 is 0.75 s, and the interval between a third beat t23 and a fourth beat t24 is 0.52 s. After the adjustment with the media editing tool, the interval between the first beat t21 and the second beat t22 of the adjusted media waveform W2 in FIG. 11 becomes 1.33 s, and the interval between the third beat t23 and the fourth beat t24 is 0.53 s. This indicates that the second tempo rate of the second media has been decreased (the interval between two beats is increased).

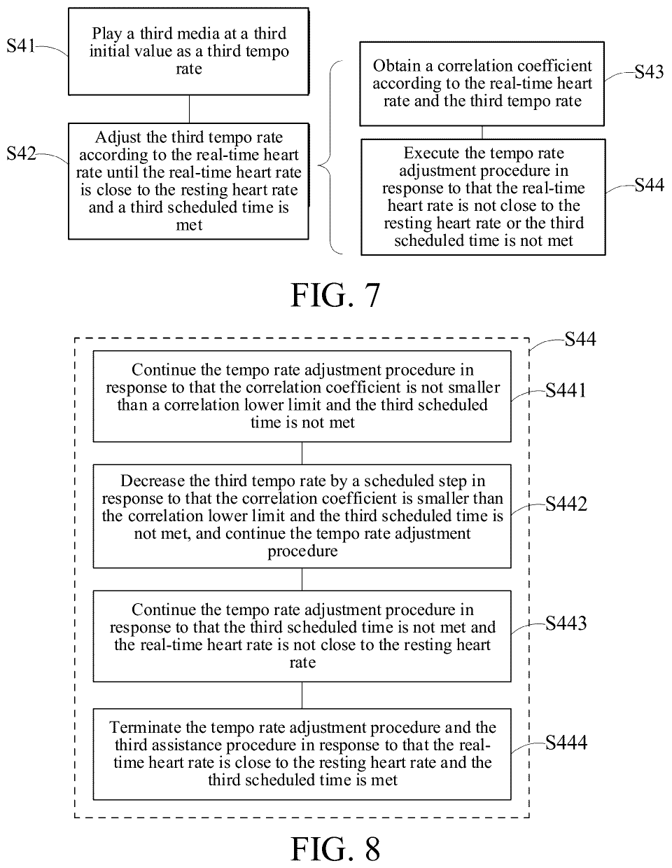

Referring to FIG. 2 and FIG. 7, FIG. 7 is a flowchart of a third assistance procedure of the sport assistance system in some embodiments of the present disclosure. As shown in FIG. 2 and FIG. 7, when the user completes the training phase (the second scheduled time is met), the real-time heart rate of the user is close to the target heart rate, and the user needs to go through a recovery phase, so that the real-time heart rate is gradually decreased to a recovery heart rate. This prevents the real-time heart rate of the user from dropping too fast, which may increase the risk of injury to the user in the long run. Therefore, in some embodiments, the processor 104 is configured to: execute the third assistance procedure according to the maximum heart rate, the resting heart rate, the third media and the real-time heart rate. The third assistance procedure includes the following steps:

Step S41: playing a third media at a third initial value as the third tempo rate; and

Step S42: adjusting the third tempo rate according to the real-time heart rate until the real-time heart rate is close to the resting heart rate and a third scheduled time is met.

The “playing the third media at the third initial value as the third tempo rate” in step S41 may mean that the sporter goes through the recovery phase after completing the training phase. In order to guide the heart rate of the user to efficiently decrease to the recovery heart rate through the third media, the processor 104 sets a third initial value of the third tempo rate to be slightly smaller than the second tempo rate (the second tempo rate corresponds to the target heart rate) at the beginning of the third assistance procedure. The third initial value refers to the initial value of the third tempo rate. By setting the third initial value to be slightly smaller than the second tempo rate, when the user does sports along with the third media, the heart rate may decrease under the influence of the third tempo rate. In some embodiments, the third initial value may be the target heart rate—the heart rate reserve*a third multiplier. The third multiplier is smaller than the second multiplier. The third multiplier may be 20%, and the third initial value is the target heart rate-20%*(the maximum heart rate—the resting heart rate).

The “adjusting the third tempo rate according to the real-time heart rate until the real-time heart rate is close to the resting heart rate and the third scheduled time is met” in step S42 may mean that as the user does cool-down exercises under the guide of the third media, when the real-time heart rate is lower than the third tempo rate, the third media may guide the heart rate of the user less effectively or ineffectively. Therefore, during the execution of the third assistance procedure, the processor 104 may decrease the third tempo rate (the third tempo rate is not greater than the real-time heart rate) in multiple phases during the decrease of the third tempo rate to keep the decreasing effect of the real-time heart rate (detailed later). It should be noted that after the processor 104 adjusts the third tempo rate, if the real-time heart rate is decreased to the third tempo rate, the user can maintain an efficient heart rate decrease rate. This helps the user quickly get into the recovery phase from the training phase in an appropriate training environment. “The real-time heart rate is close to the resting heart rate” may mean that the real-time heart rate reaches the recovery heart rate. The recovery heart rate may be 120% of the resting heart rate, or the resting heart rate+the heart rate reserve*the third multiplier (i.e., the recovery heart rate corresponds to the tempo rate of the first initial value). The third scheduled time may be a preset minimum recovery time, for example, 5 min, 6 min and 10 min.

In some embodiments, when the third assistance procedure is terminated, the third tempo rate is a third termination value, and the third termination value reaches the tempo rate corresponding to the recovery heart rate. Specifically, when the third tempo rate is close to or lower than the recovery heart rate, it indicates that the user may continue the recovery phase from the training phase. In other words, the processor 104 may end the second assistance procedure and start the third assistance procedure. When the processor 104 adjusts the third tempo rate, the interval between the third initial value and the third termination value may be divided into at least third stage, and the processor 104 may decrease the third tempo rate at each stage (detailed later). In some embodiments, the third termination value is substantially equal to the recovery heart rate (e.g., the recovery heart rate is set at 130 bpm, and the third termination value is 130 bpm).

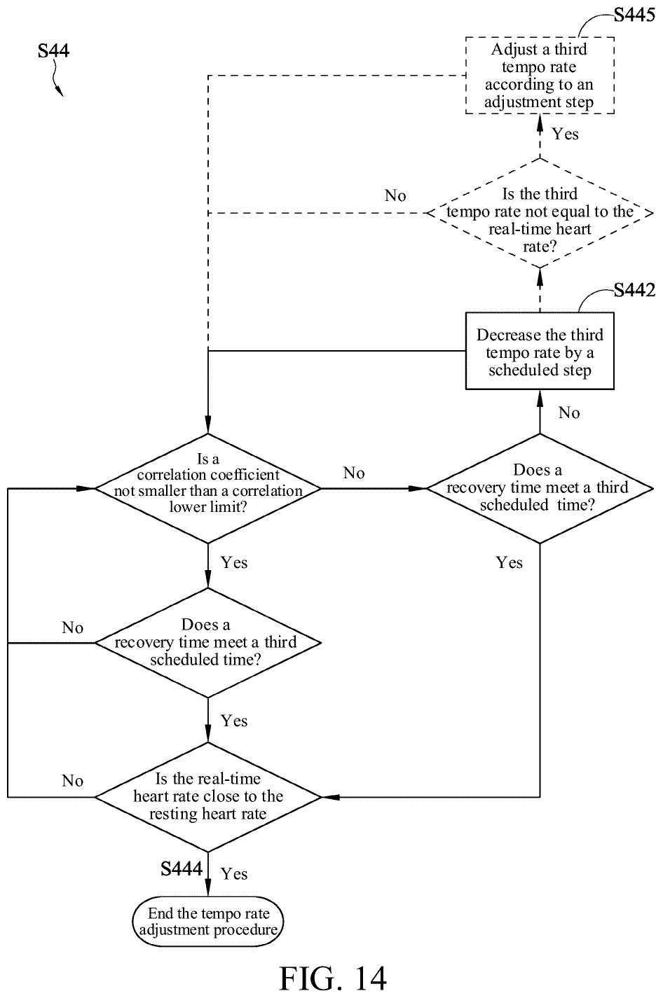

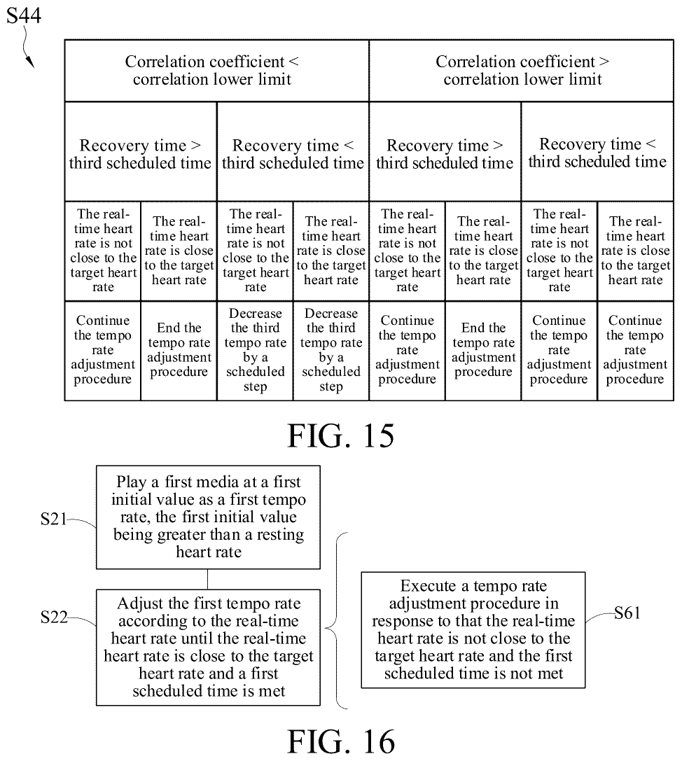

Referring to FIG. 2, FIG. 7, FIG. 8, FIG. 14 and FIG. 15, FIG. 8 is a flowchart of substeps of the third assistance procedure in FIG. 7. FIG. 14 is a flowchart of a tempo rate adjustment procedure when the sport assistance system executes the third assistance procedure in some embodiments of the present disclosure. FIG. 15 is a schematic diagram of output results of the tempo rate adjustment procedure in FIG. 14. As shown in FIG. 2, FIG. 7, FIG. 8, FIG. 14 and FIG. 15, the “adjusting the third tempo rate according to the real-time heart rate until the real-time heart rate is close to the resting heart rate and the third scheduled time is met” in step S42 further includes:

Step S43: obtaining a correlation coefficient according to the real-time heart rate and the third tempo rate; and

Step S44: executing a tempo rate adjustment procedure in response to that the real-time heart rate is not close to the resting heart rate or the third scheduled time is not met, where the tempo rate adjustment procedure includes:

Step S441: continuing the tempo rate adjustment procedure in response to that the correlation coefficient is not smaller than a correlation lower limit and the third scheduled time is not met;

Step S442: decreasing the third tempo rate by a scheduled step in response to that the correlation coefficient is smaller than the correlation lower limit and the third scheduled time is not met, and continuing the tempo rate adjustment procedure;

Step S443: continuing the tempo rate adjustment procedure in response to that the third scheduled time is not met and the real-time heart rate is not close to the resting heart rate; and

Step S444: terminating the tempo rate adjustment procedure and the third assistance procedure in response to that the real-time heart rate is close to the resting heart rate and the third scheduled time is met.

The “obtaining the correlation coefficient according to the real-time heart rate and the third tempo rate” in step S43 may mean that the processor 104 obtains the correlation coefficient between the real-time heart rate and the third tempo rate in the sliding time window. For the process of the processor 104 obtaining the correlation coefficient, reference may be made to step S23.

In the “executing the tempo rate adjustment procedure in response to that the real-time heart rate is not close to the resting heart rate or the third scheduled time is not met” in step S44, after the processor 104 obtains the correlation coefficient, if the real-time heart rate at this moment has not reached the recovery heart rate and the recovery time (the recovery time may be the start time of the third assistance procedure) has not reached the third scheduled time, the processor 104 executes the tempo rate adjustment procedure.

The “continuing the tempo rate adjustment procedure in response to that the correlation coefficient is not smaller than the correlation lower limit and the third scheduled time is not met” in step S441 may mean that after obtaining the correlation coefficient, the processor 104 compares the correlation coefficient with the correlation lower limit. If the correlation coefficient is equal to or greater than the correlation lower limit and the recovery time does not meet the third scheduled time, the processor 104 continues the tempo rate adjustment procedure (i.e., repeats the determination process of step S441). In the third assistance procedure, the correlation lower limit may be 0.8.

The “decreasing the third tempo rate by the scheduled step in response to that the correlation coefficient is smaller than the correlation lower limit and the third scheduled time is not met, and continuing the tempo rate adjustment procedure” in step S442 may mean that in a case that the processor 104 determines that the correlation coefficient is smaller than the correlation lower limit and the recovery time does not meet the third scheduled time, the processor 104 will decrease the third tempo rate by the scheduled step at this moment. For the scheduled step, reference may be made to step S242. In an example where the third initial value is 20% of the heart rate reserve and the first termination value is 50% of the heart rate reserve, the third tempo rate is decreased each time by a scheduled step of 45%*the heart rate reserve, 40%*the heart rate reserve, 35%*the heart rate reserve, 30%*the heart rate reserve, 25%*the heart rate reserve and 20%*the heart rate reserve.

Referring to FIG. 10, the following is a description in an example where the third initial value (corresponding to the tempo rate of the target heart rate) is the resting heart rate+50% the heart rate reserve (130 bpm), the recovery heart rate is the resting heart rate+20%*the heart rate reserve (100 bpm), the update frequency of the third tempo rate is once per minute and the scheduled step is set to the heart rate reserve*5%. When the third tempo rate is decreased for the first time, the third tempo rate is decreased from the resting heart rate+50%*the heart rate reserve to the resting heart rate+45%*the heart rate reserve (as shown in FIG. 10, to the tempo rate t31 in the third stage), i.e., from 130 bpm to 125 bpm (80 bpm-(180 bpm-80 bpm)*45%=125 bpm). When the third tempo rate is decreased for the second time, the third tempo rate is decreased from the resting heart rate+45%*the heart rate reserve to the resting heart rate+40%*the heart rate reserve (as shown in FIG. 10, to the tempo rate t32 in the second stage), i.e., the third tempo rate is decreased from 125 bpm to 120 bpm. When the third tempo rate is decreased for the third time, the third tempo rate is decreased from the resting heart rate+40%*the heart rate reserve to the resting heart rate+35%*the heart rate reserve (as shown in FIG. 10, to the tempo rate t33 in the third stage), i.e., the third tempo rate is decreased from 120 bpm to 115 bpm. When the third tempo rate is decreased for the fourth time, the third tempo rate is decreased from the resting heart rate+35%*the heart rate reserve to the resting heart rate+30%*the heart rate reserve (as shown in FIG. 10, to the tempo rate t34 in the fourth stage), i.e., the third tempo rate is decreased from 115 bpm to 110 bpm. When the third tempo rate is decreased for the fifth time, the third tempo rate is decreased from the resting heart rate+30%*the heart rate reserve to the resting heart rate+25%*the heart rate reserve (as shown in FIG. 10, to the tempo rate t35 in the fifth stage), i.e., the third tempo rate is decreased from 110 bpm to 105 bpm. When the third tempo rate is decreased for the sixth time, the third tempo rate is decreased from the resting heart rate+25%*the heart rate reserve to the resting heart rate+20%*the heart rate reserve (as shown in FIG. 10, to the tempo rate t36 in the sixth stage), i.e., the third tempo rate is decreased from 105 bpm to 100 bpm. In this example, the recovery heart rate is set to 100 bpm. If the real-time heart rate has been close to the recovery heart rate before the third tempo rate is decreased for the fifth time and the third scheduled time has been met, the processor 104 will not decrease the third tempo rate for the fifth time, and the processor 104 will terminate the third assistance procedure. Besides, when the third initial value is decreased to the third termination value according to the scheduled step, if the third tempo rate is updated at a frequency of once per minute, it is ensured that the third media can be played for six minutes when the processor 104 executes the third assistance procedure. In other words, the user has a recovery time of at least six minutes in the recovery phase. Thereby, the third media has enough time to guide the user to progressively decrease the sport speed and heart rate response.

The “continuing the tempo rate adjustment procedure in response to that the third scheduled time is not met and the real-time heart rate is not close to the resting heart rate” in step S443 may mean that when the recovery time does not meet the third scheduled time and the real-time heart rate is not close to the recovery heart rate, the processor 104 will repeatedly execute step S441 to determine whether the correlation coefficient is smaller than the correlation lower limit and selectively execute step S442 to decrease the third tempo rate, thereby ensuring that the third tempo rate is lower than the real-time heart rate and guiding the user to decrease the heart rate during movement.

The “terminating the tempo rate adjustment procedure and the third assistance procedure in response to that the real-time heart rate is close to the resting heart rate and the third scheduled time is met” in step S444 may mean that when determining that the real-time heart rate is smaller than or equal to the recovery heart rate and the recovery time meets the third scheduled time, the processor 104 terminates the tempo rate adjustment procedure and the third assistance procedure.

In some embodiments, the “decreasing the third tempo rate by the scheduled step in response to that the correlation coefficient is smaller than the correlation lower limit and the third scheduled time is not met, and continuing the tempo rate adjustment procedure” in step S442 further includes:

Step S445: adjusting the third tempo rate according to an adjustment step in response to that the third tempo rate lags behind the real-time heart rate, and continuing the tempo rate adjustment procedure.

The “adjusting the third tempo rate according to the adjustment step in response to that the third tempo rate lags behind the real-time heart rate, and continuing the tempo rate adjustment procedure” in step S445 may mean that the processor 104 obtains a real-time heart rate average and a third tempo rate average according to the time window. When the third tempo rate average lags behind the real-time heart rate average, it indicates that the third tempo rate lags behind or exceeds the real-time heart rate. If the correlation coefficient is smaller than the correlation lower limit and the third tempo rate lags behind the real-time heart rate, it indicates that the real-time heart rate is decreased at a rate less than expected, so that the third tempo rate is not synchronized with the real-time heart rate. The processor 104 may increase the third tempo rate by the adjustment step (i.e., the third tempo rate—the scheduled step+the adjustment step). On the contrary, if the correlation coefficient is smaller than the correlation lower limit and the third tempo rate exceeds the real-time heart rate, it indicates that the real-time heart rate is decreased at a rate more than expected, so that the third tempo rate is not synchronized with the real-time heart rate. The processor 104 may decrease the third tempo rate by the adjustment step (i.e., the third tempo rate—the scheduled step—the adjustment step). In this way, the third tempo rate can be gradually synchronized with the real-time heart rate. The adjustment step may be 5% of the real-time heart rate average. It should be noted that if the third tempo rate is still slightly greater than the real-time heart rate after the fine adjustment, the third tempo rate may be lower than the real-time heart rate in the next scheduled step, so as to ensure that the third heart rate is lower than the real-time heart rate within the recovery time. Alternatively, if the correlation coefficient is smaller than the correlation lower limit, the processor 104 may perform fine adjustment again (execute step S445 again) until the correlation coefficient is smaller than the correlation lower limit.

According to the above description, the present disclosure may provide a sport assistance method, which is applicable to the sport assistance system 10 such that the sport assistance system 10 can implement the actuation of any of the above embodiments.

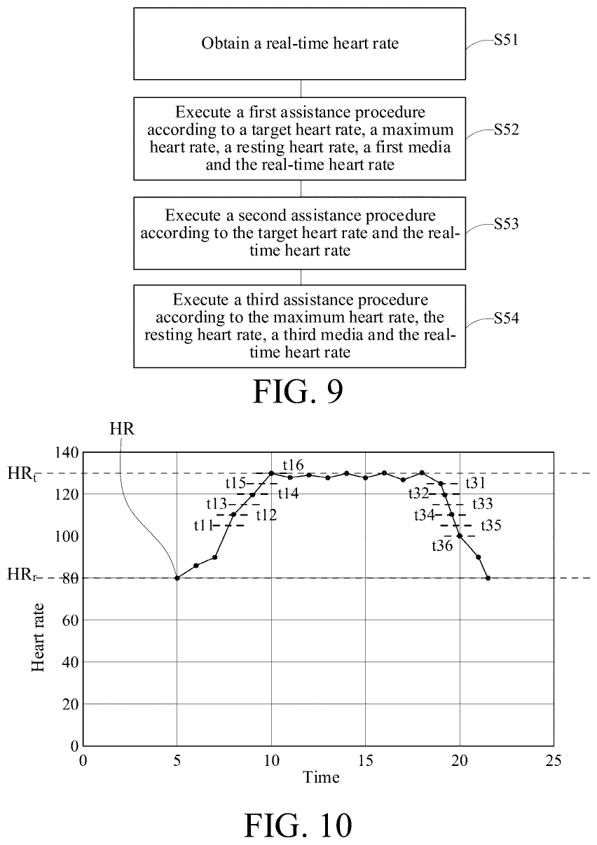

Referring to FIG. 2 and FIG. 9, FIG. 9 is a flowchart of steps of the sport assistance method in some embodiments of the present disclosure. As shown in FIG. 2 and

FIG. 9, in some embodiments, the sport assistance method includes: step S51: obtaining a real-time heart rate; step S52: executing a first assistance procedure according to a target heart rate, a maximum heart rate, a resting heart rate, a first media and the real-time heart rate; and step S53: executing a second assistance procedure according to the target heart rate and the real-time heart rate.

In this embodiment, the first assistance procedure includes: playing the first media at a first initial value as a first tempo rate, the first initial value being greater than the resting heart rate (step S21); and adjusting the first tempo rate according to the real-time heart rate until the real-time heart rate is close to the target heart rate and a first scheduled time is met (step S22). For steps S21 and S22 and substeps thereof in the first assistance procedure in the sport assistance method, reference may be made to the above descriptions of steps S23, S24, S241, S242, S243, S244 and S245 in FIG. 3 and FIG. 5.

In this embodiment, the second assistance procedure includes: playing a second media, the second media having a second tempo rate (step S31); and obtaining an adjustment parameter according to a coupling factor, and adjusting a second tempo rate according to the adjustment parameter until a second scheduled time is met (step S32). For steps S31 and S32 and substeps thereof in the second assistance procedure in the sport assistance method, reference may be made to the above descriptions of steps S31, S32, S33, S331, S332, S333 and S34 in FIG. 4 and FIG. 6.

In some embodiments, the sport assistance method further includes: step S54: executing a third assistance procedure according to the maximum heart rate, the resting heart rate, a third media and the real-time heart rate. In this embodiment, the third assistance procedure includes: playing the third media at a third initial value as the third tempo rate (step S41); and adjusting the third tempo rate according to the real-time heart rate until the real-time heart rate is close to the resting heart rate and a third scheduled time is met (step S42). For steps S41 and S42 and substeps thereof in the third assistance procedure in the sport assistance method, reference may be made to the above descriptions of any of steps S41, S42, S43, S44, S441, S442, S443, S444 and S445 in FIG. 7 and FIG. 8.

In some embodiments, heart rate data generated by the sport assistance system 10 may generate a personalized activity intelligence (PAI). The PAI calculates a quantitative evaluation score based on the quantity of exercise of the user in the past week, which serves as a quantitative indicator of exercise. The PAI can help the user to develop a habit of doing sports, and may also calculate the quantity of exercise according to the heart rate data of the user to obtain a score weight. Thus, the user can evaluate the personal motor ability according to the PAI, so as to plan a program that meets his own motor ability. The PAI may also serve as an indicator to improve health. If the PAI indicator is equal to or greater than 100, the mortality of cardiovascular diseases can be reduced by 17% in men and 23% in women. The total mortality can be reduced by 13% in men and 17% in women.

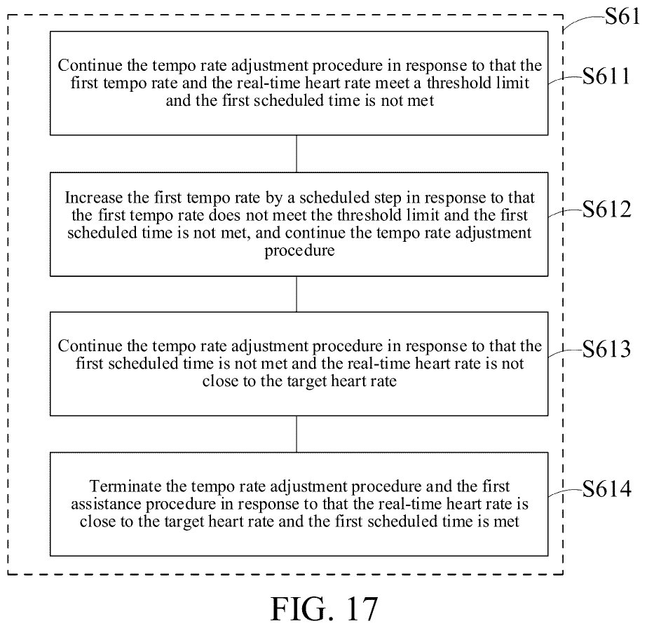

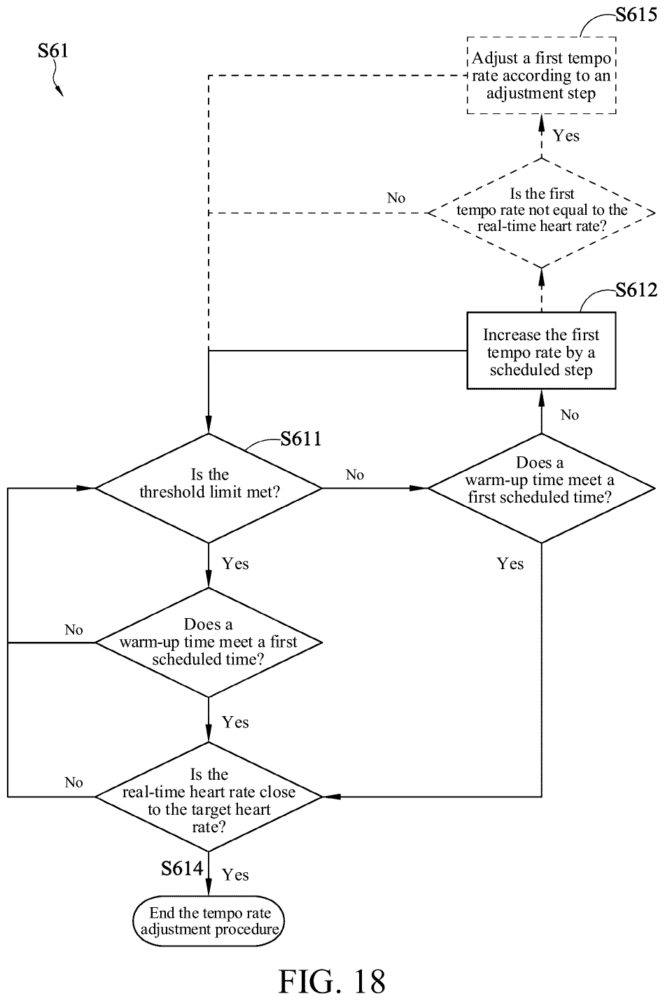

Referring to FIG. 2, FIG. 3, FIG. 16, FIG. 17 and FIG. 18, FIG. 16 is a flowchart of a first assistance procedure of the sport assistance system in other embodiments of the present disclosure. FIG. 17 is a flowchart of substeps of the first assistance procedure in FIG. 16. FIG. 18 is a flowchart of a tempo rate adjustment procedure when the sport assistance system executes the first assistance procedure in other embodiments of the present disclosure. In some embodiments, the “adjusting the first tempo rate according to the real-time heart rate until the real-time heart rate is close to the target heart rate and the first scheduled time is met” in step S22 in the first assistance procedure includes:
Step S61: executing a tempo rate adjustment procedure in response to that the real-time heart rate is not close to the target heart rate or the first scheduled time is not met, where the tempo rate adjustment procedure includes:
Step S611: continuing the tempo rate adjustment procedure in response to that the first tempo rate and the real-time heart rate meet a threshold limit and the first scheduled time is not met;
Step S612: increasing the first tempo rate by a scheduled step in response to that the first tempo rate does not meet the threshold limit and the first scheduled time is not met, and continuing the tempo rate adjustment procedure;
Step S613: continuing the tempo rate adjustment procedure in response to that the first scheduled time is not met and the real-time heart rate is not close to the target heart rate; and
Step S614: terminating the tempo rate adjustment procedure and the first assistance procedure in response to that the real-time heart rate is close to the target heart rate and the first scheduled time is met.

In step S61, when the real-time heart rate has not reached the target heart rate and the sport time has not reached the first scheduled time, the processor 104 executes the tempo rate adjustment procedure.

The “continuing the tempo rate adjustment procedure in response to that the first tempo rate and the real-time heart rate meet the threshold limit and the first scheduled time is not met” in step S611 may mean that the processor 104 may determine whether the first tempo rate and the real-time heart rate meet the threshold limit by a threshold limit method (formula (2) below). Formula (2) is as follows:

$$\vert \overline{HR} - ( a + {C}_{1} * MT ) \vert \leq {C}_{2} \qquad formula ( 2 )$$

where H̅R̅ is the real-time heart rate;
MT is the tempo rate (the first tempo rate in this example);
a is a linear intercept term constant;
C₁ is a slope term parameter; and
C₂ is a threshold parameter.

When the user is doing sports, the real-time heart rate needs to be increased along with the progressive increase of the first tempo rate that changes in stages (when the user is doing cool-down exercises, the real-time heart rate needs to be progressively decreased along with the third tempo rate that changes in stages), and the real-time heart rate and the first tempo rate (or the third tempo rate) may have a linear relationship (but not limited to the linear relationship). If the real-time heart rate keeps up with the first tempo rate, the calculated value of |H̅R̅−(α+C₁*MT) | will be small. If the calculation result is smaller than or equal to the threshold parameter C₂, it indicates that the real-time heart rate keeps up with the first tempo rate, and the second assistance procedure can be executed. However, when the sport time does not meet the first scheduled time, it indicates that the warm-up time of the user is not enough. Therefore, in this case, the processor 104 will not execute the second assistance procedure, but will continue the tempo rate adjustment procedure.

In some embodiments, the parameters C₁/(slope term parameter) and a (linear intercept term constant) may be tested before the user does sports. In the case that the sport tempo rate is progressively increased with the first tempo rate, data of the first tempo rate and the real-time heart rate are obtained, and then calculation is performed.

In some embodiments, C₂ (threshold parameter) may be set according to the motor ability of the user, and the numerical range of the threshold parameter may be from 5 bpm to 30 bpm.

The “increasing the first tempo rate by the scheduled step in response to that the first tempo rate does not meet the threshold limit and the first scheduled time is not met, and continuing the tempo rate adjustment procedure” in step S612 may mean that when the processor 104 determines that the first tempo rate and the real-time heart rate do not meet the threshold limit and the sport time does not meet the first scheduled time, the processor 104 will increase the first tempo rate by the scheduled step at this moment (step S612 is similar to step S242, and reference may be made to the description of step S242).

The “continuing the tempo rate adjustment procedure in response to that the first scheduled time is not met and the real-time heart rate is not close to the target heart rate” in step S613 may mean that when the sport time does not meet the first scheduled time and the real-time heart rate is not close to the target heart rate, the processor 104 will repeatedly execute step S611 to determine whether the first tempo rate and the real-time heart rate are smaller than or equal to the threshold limit and selectively execute step S612 to increase the first tempo rate, thereby ensuring that the first tempo rate is greater than the real-time heart rate.

The “terminating the tempo rate adjustment procedure and the first assistance procedure in response to that the real-time heart rate is close to the target heart rate and the first scheduled time is met” in step S614 may mean that when determining that the real-time heart rate is equal to or greater than the target heart rate and the sport time meets the first scheduled time, the processor 104 terminates the tempo rate adjustment procedure and the first assistance procedure and executes the second assistance procedure.

In some embodiments, the “increasing the first tempo rate by the scheduled step in response to that the first tempo rate does not meet the threshold limit and the first scheduled time is not met, and continuing the tempo rate adjustment procedure” in step S612 further includes:

Step S615: adjusting the first tempo rate according to the adjustment step in response to that the first tempo rate lags behind the real-time heart rate, and continuing the tempo rate adjustment procedure (step S615 is similar to step S245, and reference may be made to the description of step S245).

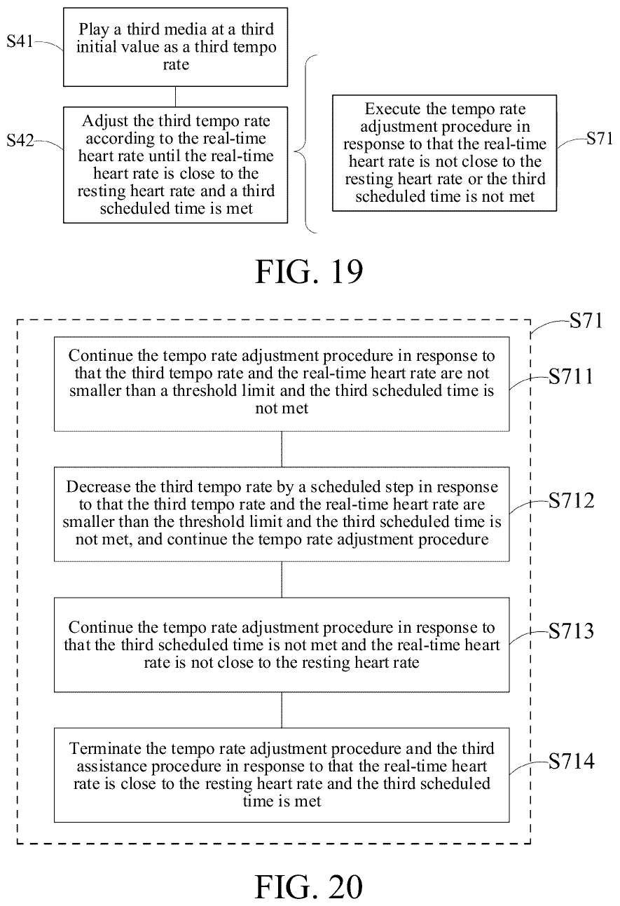

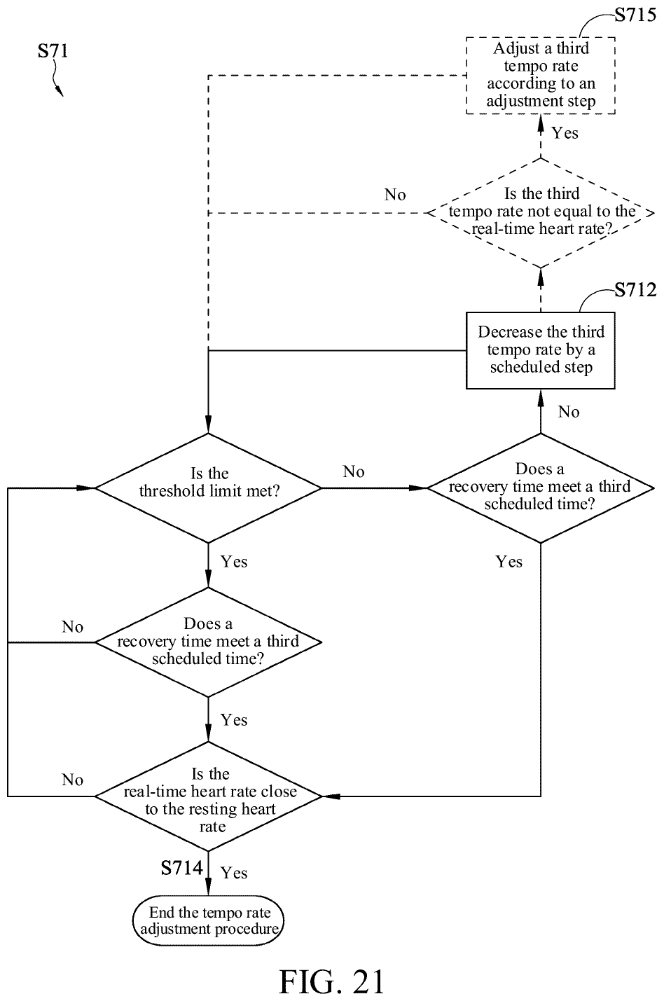

Referring to FIG. 2, FIG. 7, FIG. 8, FIG. 19, FIG. 20 and FIG. 21, FIG. 19 is a flowchart of a tempo rate adjustment procedure when the sport assistance system executes the third assistance procedure in other embodiments of the present disclosure. FIG. 20 is a schematic diagram of output results of the tempo rate adjustment procedure in FIG. 19. FIG. 21 is a flowchart of a tempo rate adjustment procedure when the sport assistance system executes the third assistance procedure in other embodiments of the present disclosure. As shown in FIG. 2, FIG. 7, FIG. 8, FIG. 19, FIG. 20 and FIG. 21, the “adjusting the third tempo rate according to the real-time heart rate until the real-time heart rate is close to the resting heart rate and the third scheduled time is met” in step S42 further includes:

Step S71: executing a tempo rate adjustment procedure in response to that the real-time heart rate is not close to the resting heart rate or the third scheduled time is not met, where the tempo rate adjustment procedure includes:

Step S711: continuing the tempo rate adjustment procedure in response to that the third tempo rate and the real-time heart rate are not smaller than a threshold limit and the third scheduled time is not met;

Step S712: decreasing the third tempo rate by a scheduled step in response to that the third tempo rate and the real-time heart rate are smaller than the threshold limit and the third scheduled time is not met, and continuing the tempo rate adjustment procedure;

Step S713: continuing the tempo rate adjustment procedure in response to that the third scheduled time is not met and the real-time heart rate is not close to the resting heart rate; and

Step S714: terminating the tempo rate adjustment procedure and the third assistance procedure in response to that the real-time heart rate is close to the resting heart rate and the third scheduled time is met.

The “executing the tempo rate adjustment procedure in response to that the real-time heart rate is not close to the resting heart rate or the third scheduled time is not met” in step S71 may mean that when the processor 104 determines the real-time heart rate has not reached the recovery heart rate at this moment and the recovery time has not reached the third scheduled time, the processor 104 executes the tempo rate adjustment procedure.

The “continuing the tempo rate adjustment procedure in response to that the third tempo rate and the real-time heart rate are not smaller than the threshold limit and the third scheduled time is not met” in step S711 may mean that when the processor 104 determines that the third tempo rate and the real-time heart rate are equal to or greater than the threshold limit and the recovery time does not meet the third scheduled time, the processor 104 continues the tempo rate adjustment procedure (i.e., repeats the determination process in step S711).

The “decreasing the third tempo rate by the scheduled step in response to that the third tempo rate and the real-time heart rate are smaller than the threshold limit and the third scheduled time is not met, and continuing the tempo rate adjustment procedure” in step S712 may mean that when the processor 104 determines that the third tempo rate and the real-time heart rate are smaller than the threshold limit and the recovery time does not meet the third scheduled time, the processor 104 decreases the third tempo rate by the scheduled step at this moment.

The “continuing the tempo rate adjustment procedure in response to that the third scheduled time is not met and the real-time heart rate is not close to the resting heart rate” in step S713 may mean that when the recovery time does not meet the third scheduled time and the real-time heart rate is not close to the recovery heart rate, the processor 104 will repeatedly execute step S711 to determine whether the third tempo rate and the real-time heart rate are smaller than the threshold limit and selectively execute step S712 to decrease the third tempo rate, thereby ensuring that the third tempo rate is lower than the real-time heart rate and guiding the user to decrease the heart rate during movement.

The “terminating the tempo rate adjustment procedure and the third assistance procedure in response to that the real-time heart rate is close to the resting heart rate and the third scheduled time is met” in step S714 may mean that when determining that the real-time heart rate is smaller than or equal to the recovery heart rate and the recovery time meets the third scheduled time, the processor 104 terminates the tempo rate adjustment procedure and the third assistance procedure.

In some embodiments, the “decreasing the third tempo rate by the scheduled step in response to that the third tempo rate and the real-time heart rate are smaller than the threshold limit and the third scheduled time is not met, and continuing the tempo rate adjustment procedure” in step S712 further includes:

Step S715: adjusting the third tempo rate according to an adjustment step in response to that the third tempo rate lags behind the real-time heart rate, and continuing the tempo rate adjustment procedure.

The “adjusting the third tempo rate according to the adjustment step in response to that the third tempo rate lags behind the real-time heart rate, and continuing the tempo rate adjustment procedure” in step S715 may mean that the processor 104 obtains a real-time heart rate average and a third tempo rate average according to the time window (step S715 is similar to step S445, and reference may be made to the description of step S445).

Based on the above, in some embodiments, the sport assistance system 10 is configured to selectively execute the first assistance procedure, the second assistance procedure or the third assistance procedure in the sport assistance method. The first assistance procedure is executed when the user just starts to do sports, and at this moment, the real-time heart rate is close to the resting heart rate. The first assistance procedure may guide the user to warm up through the first media, so that the heart rate (real-time heart rate) is increased to the target heart rate in the first scheduled time. The first tempo rate of the first media may be adjusted according to the real-time heart rate, so that the real-time heart rate is gradually increased to the target heart rate under the synchronization of the first tempo rate. The second assistance procedure may guide the user to train through the second media, so that the heart rate (real-time heart rate) is kept at the target heart rate in the second scheduled time and reaches a state of resonance and harmony, thereby improving the sport efficiency and quality of the user. The third assistance procedure may guide the user to recover through the third media, so that the heart rate is decreased to the recovery heart rate in the third scheduled time, and the third tempo rate of the third media can be adjusted according to the real-time heart rate, so that the real-time heart rate is gradually decreased to be close to the resting heart rate under the synchronization of the third tempo rate, thereby reducing the risk of injury.

The above embodiments are merely to illustrate the technical idea and characteristics of the present disclosure, and are intended to make those skilled in the art understand the contents of the present disclosure and implement them accordingly, but not to limit the scope of the present disclosure. That is, all equivalent changes or modifications made according to the spirit of the present disclosure shall fall into the scope of the present disclosure.

What is claimed is:

1. A sport assistance method, comprising:
obtaining a real-time heart rate;
executing a first assistance procedure according to a target heart rate, a maximum heart rate, a resting heart rate, a first media and the real-time heart rate, the first media having a first tempo rate, and the first assistance procedure comprising:
playing the first media at a first initial value as the first tempo rate, the first initial value being greater than the resting heart rate; and
adjusting the first tempo rate according to the real-time heart rate until the real-time heart rate is close to the target heart rate and a first scheduled time is met; and
executing a second assistance procedure according to the target heart rate and the real-time heart rate, the second assistance procedure comprising:
playing a second media, the second media having a second tempo rate; and
obtaining an adjustment parameter according to a coupling factor, and adjusting the second tempo rate according to the adjustment parameter until a second scheduled time is met.

2. The sport assistance method according to claim 1, wherein the step of adjusting the first tempo rate according to the real-time heart rate until the real-time heart rate is close to the target heart rate and the first scheduled time is met comprises:
obtaining a correlation coefficient according to the real-time heart rate and the first tempo rate; and
executing a tempo rate adjustment procedure in response to that the real-time heart rate is not close to the target heart rate or the first scheduled time is not met, the tempo rate adjustment procedure comprising:
continuing the tempo rate adjustment procedure in response to that the correlation coefficient is not smaller than a correlation lower limit and the first scheduled time is not met;
increasing the first tempo rate by a scheduled step in response to that the correlation coefficient is smaller than the correlation lower limit and the first scheduled time is not met, and continuing the tempo rate adjustment procedure;
continuing the tempo rate adjustment procedure in response to that the first scheduled time is not met and the real-time heart rate is not close to the target heart rate; and
terminating the tempo rate adjustment procedure and the first assistance procedure in response to that the real-time heart rate is close to the target heart rate and the first scheduled time is met.

3. The sport assistance method according to claim 2, wherein the step of increasing the first tempo rate by the scheduled step in response to that the correlation coefficient is smaller than the correlation lower limit and the first scheduled time is not met, and continuing the tempo rate adjustment procedure further comprises:
adjusting the first tempo rate according to an adjustment step in response to that the first tempo rate lags behind the real-time heart rate, and continuing the tempo rate adjustment procedure.

4. The sport assistance method according to claim 3, wherein the adjustment step is 5% of a real-time heart rate average.

5. The sport assistance method according to claim 2, wherein the target heart rate is the resting heart rate+a heart rate reserve*a first multiplier; the first initial value is the resting heart rate+the heart rate reserve*a second multiplier; and the scheduled step is the heart rate reserve*a third multiplier, wherein the first multiplier is greater than the second multiplier, and the third multiplier is an arithmetic progression between the first multiplier and the second multiplier.

6. The sport assistance method according to claim 1, wherein the step of obtaining the adjustment parameter according to the coupling factor, and adjusting the second tempo rate according to the adjustment parameter until the second scheduled time is met comprises:
executing a coupling factor determination procedure, the coupling factor determination procedure comprising:
obtaining the coupling factor according to a sport tempo rate and the second tempo rate;
continuing the coupling factor determination procedure when the coupling factor is smaller than a coupling factor lower limit; and
terminating the coupling factor determination procedure when the coupling factor is not smaller than the coupling factor lower limit; and
determining the adjustment parameter according to the second tempo rate, the real-time heart rate, the sport tempo rate and a tempo rate adjustment model.

7. The sport assistance method according to claim 6, wherein the tempo rate adjustment model is a Kuramoto model, the Kuramoto model comprising:
$${θ}_{t + 1 , m e n} = {ω}_{t , men} + {k}_{m e n} sin ( {θ}_{t , music} - {θ}_{t , men} ) \qquad ( 1 )$$
$${θ}_{t + 1 , m u s i c} = {ω}_{t , music} + {k}_{m u s i c} sin ( {θ}_{t , men} - {θ}_{t , music} ) \qquad ( 2 )$$
wherein θ_{t+1,men} is an angular speed of the sport tempo rate at time t+1;
θ_{1+1,music} is another angular speed of the second tempo rate at time t+1;
θ_{t,men} is a phase angle of the sport tempo rate at time t;
θ_{t,music} is another phase angle of the second tempo rate at time t;
ω_{t,men} is an initial angular speed of the sport tempo rate at time t;
ω_{t,music} is another initial angular speed of the second tempo rate at time t;
kₘₑₙ is a coupling constant of the sport tempo rate; and
k_{music} is another coupling constant of the second tempo rate.

8. The sport assistance method according to claim 6, wherein the second tempo rate is transformed to a phase plane to obtain a first phase angle; the sport tempo rate is transformed to the phase plane to obtain a second phase angle; the coupling factor is obtained according to the first phase angle, the second phase angle and a coupling factor formula.

9. The sport assistance method according to claim 8, wherein the coupling factor formula is: ∥F1+F2∥/∥F1∥+∥F2∥, wherein F1 is the first phase angle; F2 is the second phase angle; ∥F1∥ is the size of the first phase angle; and ∥F2∥ is the size of the second phase angle.

10. The sport assistance method according to claim 1, further comprising: executing a third assistance procedure according to the maximum heart rate, the resting heart rate, a third media and the real-time heart rate, the third media having a third tempo rate, and the third assistance procedure comprising:
playing the third media at a third initial value as the third tempo rate; and
adjusting the third tempo rate according to the real-time heart rate until the real-time heart rate is close to the resting heart rate and a third scheduled time is met.

11. The sport assistance method according to claim 10, wherein the step of adjusting the third tempo rate according to the real-time heart rate until the real-time heart rate is close to the resting heart rate and the third scheduled time is met further comprises:
obtaining a correlation coefficient according to the real-time heart rate and the third tempo rate; and
executing a tempo rate adjustment procedure in response to that the real-time heart rate is not close to the resting heart rate or the third scheduled time is not met, the tempo rate adjustment procedure comprising:
continuing the tempo rate adjustment procedure in response to that the correlation coefficient is not smaller than a correlation lower limit and the third scheduled time is not met;
decreasing the third tempo rate by a scheduled step in response to that the correlation coefficient is smaller than the correlation lower limit and the third scheduled time is not met, and continuing the tempo rate adjustment procedure;
continuing the tempo rate adjustment procedure in response to that the third scheduled time is not met and the real-time heart rate is not close to the resting heart rate; and
terminating the tempo rate adjustment procedure and the third assistance procedure in response to that the real-time heart rate is close to the resting heart rate and the third scheduled time is met.

12. The sport assistance method according to claim 11, wherein the step of decreasing the third tempo rate by the scheduled step in response to that the correlation coefficient is smaller than the correlation lower limit and the third scheduled time is not met, and continuing the tempo rate adjustment procedure comprises:
adjusting the third tempo rate according to an adjustment step in response to that the third tempo rate lags behind the real-time heart rate, and continuing the tempo rate adjustment procedure.

13. The sport assistance method according to claim 12, wherein the target heart rate is the resting heart rate+a heart rate reserve*a first multiplier; the first initial value is the resting heart rate+the heart rate reserve*a second multiplier; and the scheduled step is the heart rate reserve*a third multiplier, wherein the first multiplier is greater than the second multiplier, and the third multiplier is an arithmetic progression between the first multiplier and the second multiplier.

14. A sport assistance system, comprising:
a heart rate sensor, configured to obtain a real-time heart rate; and
a processor, configured to:
execute a first assistance procedure according to a target heart rate, a maximum heart rate, a resting heart rate, a first media and the real-time heart rate, the first media having a first tempo rate, and the first assistance procedure comprising:
playing the first media at a first initial value as the first tempo rate, the first initial value being greater than the resting heart rate; and
adjusting the first tempo rate according to the real-time heart rate until the real-time heart rate is close to the target heart rate and a first scheduled time is met; and
execute a second assistance procedure according to the target heart rate and the real-time heart rate, the second assistance procedure comprising:
playing a second media, the second media having a second tempo rate; and
obtaining an adjustment parameter according to a coupling factor, and adjusting the second tempo rate according to the adjustment parameter until a second scheduled time is met.

15. The sport assistance system according to claim 14, wherein the step of adjusting the first tempo rate according to the real-time heart rate until the real-time heart rate is close to the target heart rate and the first scheduled time is met by the processor further comprises:
obtaining a correlation coefficient according to the real-time heart rate and the first tempo rate;
executing a tempo rate adjustment procedure in response to that the real-time heart rate is not close to the target heart rate or the first scheduled time is not met, the tempo rate adjustment procedure comprising:
continuing the tempo rate adjustment procedure in response to that the correlation coefficient is not smaller than a correlation lower limit and the first scheduled time is not met;
increasing the first tempo rate by a scheduled step in response to that the correlation coefficient is smaller than the correlation lower limit and the first scheduled time is not met, and continuing the tempo rate adjustment procedure;
continuing the tempo rate adjustment procedure in response to that the first scheduled time is not met and the real-time heart rate is not close to the target heart rate; and
terminating the tempo rate adjustment procedure and the first assistance procedure in response to that the real-time heart rate is close to the target heart rate and the first scheduled time is met.

16. The sport assistance system according to claim 15, wherein the step of increasing the first tempo rate by the scheduled step in response to that the correlation coefficient is smaller than the correlation lower limit and the first scheduled time is not met, and continuing the tempo rate adjustment procedure by the processor further comprises:
adjusting the first tempo rate according to an adjustment step in response to that the first tempo rate lags behind the real-time heart rate, and continuing the tempo rate adjustment procedure.

17. The sport assistance system according to claim 15, wherein the target heart rate is the resting heart rate+a heart rate reserve*a first multiplier; the first initial value is the resting heart rate+the heart rate reserve*a second multiplier; and the scheduled step is the heart rate reserve*a third multiplier, wherein the first multiplier is greater than the second multiplier, and the third multiplier is an arithmetic progression between the first multiplier and the second multiplier.

18. The sport assistance system according to claim 14, wherein the step of obtaining the adjustment parameter according to the coupling factor, and adjusting the second tempo rate according to the adjustment parameter until the second scheduled time is met by the processor further comprises:
executing a coupling factor determination procedure, the coupling factor determination procedure comprising:
obtaining the coupling factor according to a sport tempo rate and the second tempo rate;
continuing the coupling factor determination procedure when the coupling factor is smaller than a coupling factor lower limit; and
terminating the coupling factor determination procedure when the coupling factor is not smaller than the coupling factor lower limit; and
determining the adjustment parameter according to the second tempo rate, the real-time heart rate, the sport tempo rate and a tempo rate adjustment model.

19. The sport assistance system according to claim 14, wherein the processor is further configured to: execute a third assistance procedure according to the maximum heart rate, the resting heart rate, a third media and the real-time heart rate, the third media having a third tempo rate, and the third assistance procedure comprising:
executing a coupling factor determination procedure while executing the second assistance procedure, the coupling factor determination procedure comprising:
playing the third media at a third initial value as the third tempo rate; and
adjusting the third tempo rate according to the real-time heart rate until the real-time heart rate is close to the resting heart rate and a third scheduled time is met.

20. The sport assistance system according to claim 19, wherein the step of adjusting the third tempo rate according to the real-time heart rate until the real-time heart rate is close to the resting heart rate and the third scheduled time is met by the processor further comprises:
obtaining a correlation coefficient according to the real-time heart rate and the third tempo rate; and
executing a tempo rate adjustment procedure in response to that the real-time heart rate is not close to the resting heart rate or the third scheduled time is not met, the tempo rate adjustment procedure comprising:
continuing the tempo rate adjustment procedure in response to that the correlation coefficient is not smaller than a correlation lower limit and the third scheduled time is not met;
decreasing the third tempo rate by a scheduled step in response to that the correlation coefficient is smaller than the correlation lower limit and the third scheduled time is not met, and continuing the tempo rate adjustment procedure;
continuing the tempo rate adjustment procedure in response to that the third scheduled time is not met and the real-time heart rate is not close to the resting heart rate; and
terminating the tempo rate adjustment procedure and the third assistance procedure in response to that the real-time heart rate is close to the resting heart rate and the third scheduled time is met.
